SEQUENCE LISTING (10 sequences)

SEQ ID NO: 1 - DNA, 3162 bp, synthetic construct
  source 1..3162: mol_type = other DNA
atgagcagcgcgatcaaaagctacaagagcgttctgcgtccgaacgagcgtaagaaccaactgctgaaaagcaccattcagtgcctggaagacggtagcgcgttctttttcaagatgctgcaaggcctgtttggtggcatcaccccggagattgttcgtttcagcaccgaacaggagaaacagcaacaggatatcgcgctgtggtgcgcggttaactggttccgtccggtgagccaagacagcctgacccacaccattgcgagcgataacctggtggagaagtttgaggaatactatggtggcaccgcgagcgacgcgatcaaacagtacttcagcgcgagcattggcgaaagctactattggaacgactgccgtcaacagtactatgatctgtgccgtgagctgggtgttgaggtgagcgacctgacccatgatctggagatcctgtgccgtgaaaagtgcctggcggttgcgaccgagagcaaccagaacaacagcatcattagcgttctgtttggcaccggcgaaaaagaggaccgtagcgtgaaactgcgtatcaccaagaaaattctggaggcgatcagcaacctgaaagaaatcccgaagaacgttgcgccgattcaagagatcattctgaacgtggcgaaagcgaccaaggaaaccttccgtcaggtgtatgcgggtaacctgggtgcgccgagcaccctggagaaatttatcgcgaaggacggccaaaaagagttcgatctgaagaaactgcagaccgacctgaagaaagttattcgtggtaaaagcaaggagcgtgattggtgctgccaggaagagctgcgtagctacgtggagcaaaacaccatccagtatgacctgtgggcgtggggcgaaatgttcaacaaagcgcacaccgcgctgaaaatcaagagcacccgtaactacaactttgcgaagcaacgtctggaacagttcaaagagattcagagcctgaacaacctgctggttgtgaagaagctgaacgactttttcgatagcgaatttttcagcggcgaggaaacctacaccatctgcgttcaccatctgggtggcaaggacctgagcaaactgtataaggcgtgggaggatgatccggcggacccggaaaacgcgattgtggttctgtgcgacgatctgaaaaacaactttaagaaagagccgatccgtaacattctgcgttacatcttcaccattcgtcaagaatgcagcgcgcaggacatcctggcggcggcgaagtacaaccaacagctggatcgttataaaagccaaaaggcgaacccgagcgttctgggtaaccagggctttacctggaccaacgcggtgatcctgccggagaaggcgcagcgtaacgaccgtccgaacagcctggatctgcgtatttggctgtacctgaaactgcgtcacccggacggtcgttggaagaaacaccatatcccgttctacgatacccgtttcttccaagaaatttatgcggcgggcaacagcccggttgacacctgccagtttcgtaccccgcgtttcggttatcacctgccgaaactgaccgatcagaccgcgatccgtgttaacaagaaacatgtgaaagcggcgaagaccgaggcgcgtattcgtctggcgatccaacagggcaccctgccggtgagcaacctgaagatcaccgaaattagcgcgaccatcaacagcaaaggtcaagtgcgtattccggttaagtttgacgtgggtcgtcaaaaaggcaccctgcagatcggtgaccgtttctgcggctacgatcaaaaccagaccgcgagccacgcgtatagcctgtgggaagtggttaaagagggtcaataccataaagagctgggctgctttgttcgtttcatcagcagcggtgacatcgtgagcattaccgagaaccgtggcaaccaatttgatcagctgagctatgaaggtctggcgtacccgcaatatgcggactggcgtaagaaagcgagcaagttcgtgagcctgtggcagatcaccaagaaaaacaagaaaaaggaaatcgtgaccgttgaagcgaaagagaagtttgacgcgatctgcaagtaccagccgcgtctgtataaattcaacaaggagtacgcgtatctgctgcgtgatattgttcgtggcaaaagcctggtggaactgcaacagattcgtcaagagatctttcgtttcattgaacaggactgcggtgttacccgtctgggcagcctgagcctgagcaccctggaaaccgtgaaagcggttaagggtatcatttacagctattttagcaccgcgctgaacgcgagcaagaacaacccgatcagcgacgaacagcgtaaagagtttgatccggaactgttcgcgctgctggaaaagctggagctgattcgtacccgtaaaaagaaacaaaaagtggaacgtatcgcgaacagcctgattcagacctgcctggagaacaacatcaagttcattcgtggtgaaggcgacctgagcaccaccaacaacgcgaccaagaaaaaggcgaacagccgtagcatggattggttggcgcgtggtgtttttaacaaaatccgtcaactggcgccgatgcacaacattaccctgttcggttgcggcagcctgtacaccagccaccaggacccgctggtgcatcgtaacccggataaagcgatgaagtgccgttgggcggcgatcccggttaaggacattggcgattgggtgctgcgtaagctgagccaaaacctgcgtgcgaaaaacatcggcaccggcgagtactatcaccaaggtgttaaagagttcctgagccattatgaactgcaggacctggaggaagagctgctgaagtggcgtagcgatcgtaaaagcaacattccgtgctgggtgctgcagaaccgtctggcggagaagctgggcaacaaagaagcggtggtttacatcccggttcgtggtggccgtatttattttgcgacccacaaggtggcgaccggtgcggtgagcatcgttttcgaccaaaaacaagtgtgggtttgcaacgcggatcatgttgcggcggcgaacatcgcgctgaccgtgaagggtattggcgaacaaagcagcgacgaagagaacccggatggtagccgtatcaaactgcagctgaccagc

SEQ ID NO: 2 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 3 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGRWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 4 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 5 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGGRIKLQLTS

SEQ ID NO: 6 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 7 - protein, 1054 aa, synthetic construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 8 - DNA, 5114 bp, Homo sapiens
  source 1..5114: mol_type = other DNA
aacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttcattaaaatgatttggaagagcaga

SEQ ID NO: 9 - DNA, 7692 bp, Homo sapiens
  source 1..7692: mol_type = other DNA
acttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcatttttttttaagaaaaacgcctgccttctgcgtgagattctccagagcaaactgggcggcatgggccctgtggtcttttcgtacagagggcttcctctttggctctttgcctggttgtttccaagatgtactgtgcctcttactttcggttttgaaaacatgagggggttgggcgtggtagcttacgcctgtaatcccagcacttagggaggccgaggcgggaggatggcttgaggtccgtagttgagaccagcctggccaacatggtgaagcctggtctctacaaaaaataataacaaaaattagccgggtgtggtggctcgtgcctgtggtcccagctgctccggtggctgaggcgggaggatctcttgagcttaggcttttgagctatcatggcgccagtgcactccagcgtgggcaacagagcgagaccctgtctctcaaaaaagaaaaaaaaaaaaaaagaaagagaaaagaaaagaaagaaagaagtgaaggtttgtcagtcaggggagctgtaaaaccattaataaagataatccaagatggttaccaagactgttgaggacgccagagatcttgagcactttctaagtacctggcaatacactaagcgcgctcaccttttcctctggcaaaacatgatcgaaagcagaatgttttgatcatgagaaaattgcatttaatttgaatacaatttatttacaacataaaggataatgtatatatcaccaccattactggtatttgctggttatgttagatgtcattttaaaaaataacaatctgatatttaaaaaaaaatcttattttgaaaatttccaaagtaatacatgccatgcatagaccatttctggaagataccacaagaaacatgtaatgatgattgcctctgaaggtctattttcctcctctgacctgtgtgtgggttttgtttttgttttactgtgggcataaattaatttttcagttaagttttggaagcttaaataactctccaaaagtcataaagccagtaactggttgagcccaaattcaaacccagcctgtctgatacttgtcctcttcttagaaaagattacagtgatgctctcacaaaatcttgccgccttccctcaaacagagagttccaggcaggatgaatctgtgctctgatccctgaggcatttaatatgttcttattattagaagctcagatgcaaagagctctcttagcttttaatgttatgaaaaaaatcaggtcttcattagattccccaatccacctcttgatggggctagtagcctttccttaatgatagggtgtttctagagagatatatctggtcaaggtggcctggtactcctccttctccccacagcctcccagacaaggaggagtagctgccttttagtgatcatgtaccctgaatataagtgtatttaaaagaattttatacacatatatttagtgtcaatctgtatatttagtagcactaacacttctcttcattttcaatgaaaaatatagagtttataatattttcttcccacttccccatggatggtctagtcatgcctctcattttggaaagtactgtttctgaaacattaggcaatatattcccaacctggctagtttacagcaatcacctgtggatgctaattaaaacgcaaatcccactgtcacatgcattactccatttgatcataatggaaagtatgttctgtcccatttgccatagtcctcacctatccctgttgtattttatcgggtccaactcaaccatttaaggtatttgccagctcttgtatgcatttaggttttgtttctttgttttttagctcatgaaattaggtacaaagtcagagaggggtctggcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctcttatatctcactcccactattacccctttattttcaaacagggaaacagtcttcaagttccacttggtaaaaaatgtgaaccccttgtatatagagtttggctcacagtgtaaagggcctcagtgattcacattttccagattaggaatctgatgctcaaagaagttaaatggcatagttggggtgacacagctgtctagtgggaggccagccttctatattttagccagcgttctttcctgcgggccaggtcatgaggagtatgcagactctaagagggagcaaaagtatctgaaggatttaatattttagcaaggaatagatatacaatcatcccttggtctccctgggggattggtttcaggaccccttcttggacaccaaatctatggatatttaagtcccttctataaaatggtatagtatttgcatataacctatccacatcctcctgtatactttaaatcatttctagattacttgtaatacctaatacaatgtaaatgctatgcaaatagttgttattgtttaaggaataatgacaagaaaaaaaagtctgtacatgctcagtaaagacacaaccatccctttttttccccagtgtttttgatccatggtttgctgaatccacagatgtggagcccctggatacggaaggcccgctgtactttgaatgacaaataacagatttaaaattttcaaggcatagttttatacctga

SEQ ID NO: 10 - DNA, 5536 bp, synthetic construct
  source 1..5536: mol_type = other DNA
aggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtacagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatgctcaggctgctcttggctctcaacttattcccttcaattcaagtaacaggagggtcttcggactacaaggatcatgacggagactataaggatcacgatattgattacaaagatgacgacgacaaagacatccagatgacacagactacatcctccctgtctgcctctctgggagacagagtcaccatcagttgcagggcaagtcaggacatctctaagtatttgaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaggtaccaccacaacgcccgctcctcggccaccgacgccagcgccaactattgcgagtcagcctctcagtctgcgacctgaggcttgtcgaccagcagccggaggcgcagtgcacacgagggggctggacttcgcctgtgatagaagacctccttctaagcccttttgggtgctggtggtggttggtggagtcctggcttgctatagcttgctagtaacagtggcctttattattttctgggtgaggaaacggggcagaaagaaactcctgtatatattcaaacaaccatttatgagaccagtacaaactactcaagaggaagatggctgtagctgccgatttccagaagaagaagaaggaggatgtgaactggctagcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctagctaacagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgtggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctgcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgaca

(19) United States
(12) Patent Application Publication
(10) Pub. No.: US 2024/0368632 A1
(43) Pub. Date: Nov. 7, 2024

(54) CELLS MODIFIED BY A CAS12I POLYPEPTIDE

(71) Applicant: Arbor Biotechnologies, Inc., Cambridge, MA (US)

(72) Inventors: Tia Marie DiTommaso, Newton, MA (US); Jeffrey Raymond Haswell, Needham, MA (US); Noah Michael Jakimo, Cambridge, MA (US)

(73) Assignee: Arbor Biotechnologies, Inc., Cambridge, MA (US)

(21) Appl. No.: 18/688,176

(22) PCT Filed: Sep. 1, 2022
(86) PCT No.: PCT/US2022/042298

§ 371 Date: Feb. 29, 2024

Related U.S. Application Data
(60) Provisional application No. 63/239,477, filed on Sep. 1, 2021.

(51) Int. Cl. C12N 15/90; C07K 14/725; C12N 5/0783; C12N 9/22; C12N 15/11; C12N 15/86

(52) U.S. Cl. CPC C12N 15/907; C07K 14/7051; C12N 5/0636; C12N 9/22; C12N 15/111; C12N 15/86; C07K 2317/622; C07K 2319/02; C07K 2319/03; C12N 2310/20; C12N 2510/00; C12N 2740/15043; C12N 2750/14143

(57) ABSTRACT

The present disclosure relates to cells (e.g., T cells) modified by Cas12i, methods of modifying the cells, processes for characterizing the modified cells, compositions and formulations comprising the modified cells, and uses of the compositions and formulations comprising the modified cells.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY

It is against the above background that the present disclosure provides certain advantages and advancements over the prior art. Although this invention disclosed herein is not limited to specific advantages or functionalities, the invention provides a method of making a modified T cell, the method comprising introducing into a T cell: (a) a variant Cas12i polypeptide or a nucleic acid encoding the variant Cas12i polypeptide, (b) a first RNA guide or a nucleic acid encoding the first RNA guide, and (c) an exogenous nucleic acid for integration into the genome of the T cell.

The invention further provides a method of making a modified T cell, the method comprising: (a) disrupting a gene in the genome of a T cell using a variant Cas12i polypeptide and a first RNA guide, and (b) introducing an exogenous nucleic acid into the genome of the T cell.

In one aspect of the methods, the variant Cas12i polypeptide is a variant Cas12i2 polypeptide.

In another aspect of the methods, the variant Cas12i polypeptide comprises a sequence having at least 90% identity to a sequence of any one of SEQ ID NOs: 3-7.

In another aspect of the methods, the variant Cas12i polypeptide comprises a sequence of any one of SEQ ID NOs: 3-7.

In another aspect of the methods, the first RNA guide comprises a spacer sequence specific to a TRAC gene or to a B2M gene.

In another aspect of the methods, the TRAC gene or the B2M gene is disrupted.

In another aspect of the methods, the disruption is a deletion.

In another aspect of the methods, the disruption is an insertion.

In another aspect of the methods, the disruption occurs in one or both alleles of the TRAC gene or the B2M gene.

In another aspect of the methods, the disruption inhibits or decreases expression of the TRAC gene or the B2M gene.

In another aspect of the methods, the disruption is within 100 nucleotides of a 5′-NTTN-3′ sequence.

In another aspect of the methods, the exogenous nucleic acid is integrated into the genome.

In another aspect of the methods, the exogenous nucleic acid is integrated into the TRAC gene or the B2M gene.

In another aspect of the methods, the exogenous nucleic acid comprises a sequence encoding a protein.

In another aspect of the methods, the exogenous nucleic acid comprises a regulatory sequence operably linked to the sequence encoding the protein.

In another aspect of the methods, the protein is a chimeric antigen receptor (CAR).

In another aspect of the methods, the CAR comprises a single chain antibody (scFv), a transmembrane domain, an intracellular signaling domain, and, optionally, a hinge domain between the scFv and the transmembrane domain.

In another aspect of the methods, the intracellular signaling domain comprises a CD3ζ signaling domain.

In another aspect of the methods, the intracellular signaling domain further comprises a signaling domain selected from the group consisting of CD28, 4-1BB, ICOS, OX40, CD27, p56-lck, or a combination of two or more thereof.

In another aspect of the methods, the CAR comprises a scFv that is specific for CD19, CD22, BCMA, HER2, IL13Ra2, CD123, FAP, VEGFR-2, ganglioside GD2, EGFRvIII, mesothelin, or EphA.

In another aspect of the methods, the exogenous nucleic acid is integrated into the genome of the T cell by homology directed repair (HDR).

In another aspect of the methods, the exogenous nucleic acid is included in a vector.

In another aspect of the methods, the vector is a viral vector.

In another aspect of the methods, the viral vector is a lentiviral vector or an AAV vector.

In another aspect of the methods, the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the TRAC gene.

In another aspect of the methods, the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the B2M gene.

The invention yet further provides a modified T cell generated by a method described herein.

In one aspect of the modified T cell, the T cell is a human T cell or is derived from a human cell or a human T cell.

The invention yet further provides a composition or formulation comprising one or more modified T cell(s) of described herein.

The invention yet further provides a method of making a modified T cell, the method comprising introducing into a T cell: (a) a variant Cas12i polypeptide, or a nucleic acid encoding the variant Cas12i polypeptide, and a first RNA guide, or a nucleic acid encoding the first RNA guide, for inducing a deletion or an insertion in the genome of the T cell; and (b) an exogenous nucleic acid for integration into the genome of the T cell.

The invention yet further provides a method of making a modified T cell, the method comprising: (a) introducing a deletion or an insertion into the genome of a T cell using a variant Cas12i polypeptide and a first RNA guide; and (b) introducing an exogenous nucleic acid into the genome of the T cell.

In one aspect of the methods, the variant Cas12i polypeptide is a variant Cas12i2 polypeptide.

In another aspect of the methods, the variant Cas12i polypeptide comprises a sequence having at least 90% identity to a sequence of any one of SEQ ID NOs: 3-7.

In another aspect of the methods, the variant Cas12i polypeptide comprises a sequence of any one of SEQ ID NOs: 3-7.

In another aspect of the methods, the integration of the exogenous nucleic acid is within 100 nucleotides of a 5′-NTTN-3′ sequence.

In another aspect of the methods, the first RNA guide comprises a spacer sequence specific to a TRAC gene or to a B2M gene.

In another aspect of the methods, the deletion or the insertion is introduced into the TRAC gene or the B2M gene.

In another aspect of the methods, the deletion or the insertion is in one or both alleles of the TRAC gene or the B2M gene.

In another aspect of the methods, the deletion or the insertion inhibits or decreases expression of the TRAC gene or the B2M gene.

In another aspect of the methods, the deletion or the insertion is within 100 nucleotides of a 5′-NTTN-3′ sequence.

In another aspect of the methods, the exogenous nucleic acid is integrated into the TRAC gene or the B2M gene.

In another aspect of the methods, the exogenous nucleic acid comprises a sequence encoding a protein.

In another aspect of the methods, the exogenous nucleic acid comprises a regulatory sequence operably linked to the sequence encoding the protein.

In another aspect of the methods, the protein is a chimeric antigen receptor (CAR).

In another aspect of the methods, the CAR comprises a single chain antibody (scFv), a transmembrane domain, an intracellular signaling domain, and, optionally, a hinge domain between the scFv and the transmembrane domain.

In another aspect of the methods, the intracellular signaling domain comprises a CD3ζ signaling domain.

In another aspect of the methods, the intracellular signaling domain further comprises a signaling domain selected from the group consisting of CD28, 4-1BB, ICOS, OX40, CD27, p56-lck, or a combination of two or more thereof.

In another aspect of the methods, the CAR comprises a scFv that is specific for CD19, CD22, BCMA, HER2, IL13Ra2, CD123, FAP, VEGFR-2, ganglioside GD2, EGFRvIII, mesothelin, or EphA.

In another aspect of the methods, the exogenous nucleic acid is integrated into the genome of the T cell by homology directed repair (HDR).

In another aspect of the methods, the exogenous nucleic acid is included in a vector.

In another aspect of the methods, the vector is a viral vector.

In another aspect of the methods, the viral vector is a lentiviral vector or an AAV vector.

In another aspect of the methods, the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the TRAC gene.

In another aspect of the methods, the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the B2M gene.

The invention yet further provides a modified T cell generated by a method described herein.

In one aspect of the modified T cell, the T cell is a human T cell or is derived from a human cell or a human T cell.

The invention yet further provides a composition or formulation comprising one or more modified T cell(s) described herein.

### Definitions

The present disclosure will be described with respect to particular embodiments and with reference to certain Figures, but the disclosure is not limited thereto but only by the claims. Terms as set forth hereinafter are generally to be understood in their common sense unless indicated otherwise.

As used herein, the term “adjacent to” refers to a sequence in close proximity to another sequence within the same nucleic acid molecule (e.g., DNA). In some embodiments, a sequence is adjacent to another sequence if no nucleotides separate the two sequences. In some embodiments, a sequence is adjacent to another sequence if a small number of nucleotides separate the two sequences (e.g., about 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, or 200 nucleotides). In some embodiments, a first sequence is adjacent to a second sequence if the two sequences are separated by about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides. As used herein, the term “adjacent to” is used to refer to the positioning of an insertion in a modified cell of the disclosure. In some embodiments, the positioning of the insertion is relative to a 5′-NTTN-3′ as described herein. In some embodiments, the sequence adjacent to another sequence is upstream of the other sequence. In some embodiments, the sequence adjacent to another sequence is downstream of the other sequence.

As used herein, the term “CAR” or “chimeric antigen receptor” refers to a protein that includes an extracellular antigen binding domain (e.g., a single chain antibody (scFv)) fused to a cell membrane proximal spacer or hinge domain, which in turn is fused to a transmembrane domain (e.g., a CD28 transmembrane domain), followed by an intracellular T cell signaling domain (e.g., an immunoreceptor tyrosine-based activation motif (ITAM)-containing domain such as, e.g., CD3-ξ). The term includes first generation CARs, which include only an intracellular T cell signaling domain (e.g., CD3-ξ) in their intracellular portion, as well as second and third generation CARs, which also include one or more than one intracellular co-stimulatory domain (e.g., CD28, CD27, CD134 (OX40), ICOS, p56-lck, and/or CD137 (4-1BB) intracellular co-stimulatory domains), respectively. Fourth generation CARs, which are second generation CARs that also include one or more factors that enhance T cell expansion, persistence, and antitumoral activity (e.g., a cytokine, such as IL-2, IL-5, IL-12, and/or a co-stimulatory ligand) are also included. Further, fifth generation CARs, which are second generation CARs that also include an intracellular domain of a cytokine receptors (e.g. an IL-2Rβ chain fragment) are included within the term. In addition, the term includes multi-specific CARs (e.g., bi-specific CARs) and split CARs. The term also includes CARs in which the spacer or hinge domain is not present. It is to be understood that when mention is made herein of the insertion of a CAR sequence, the insertion can also optionally include a regulatory sequence (e.g., a promoter) that directs expression of the CAR, unless the context indicates otherwise.

As used herein, the term “Cas12i polypeptide” (also referred to herein as Cas12i) refers to a polypeptide comprising at least 70%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 1-5 and SEQ ID NOs: 11-18 of U.S. Pat. No. 10,808,245, which is incorporated by reference herein in its entirety. In some embodiments, a Cas12i polypeptide comprises at least 70%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 3, 5, 14, or 16 of U.S. Pat. No. 10,808,245. In some embodiments, the Cas12i polypeptide of the disclosure is a Cas12i2 polypeptide as described in PCT/US2021/025257 (WO 2021/202800). In some embodiments, a Cas12i2 polypeptide comprises at least 70%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 3-7. In some embodiments, a Cas12i2 polypeptide comprises a sequence of any one of SEQ ID NOs: 3-7. In some embodiments, a Cas12i2 polypeptide of the disclosure is encoded by a nucleic acid sequence comprising at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with SEQ ID NO: 1. In some embodiments, a Cas12i2 polypeptide of the disclosure is encoded by a nucleic acid sequence comprising the sequence of SEQ ID NO: 1.

As used herein, the terms “variant Cas12i2 polypeptide” and “variant effector polypeptide” refer to a polypeptide comprising an alteration, e.g., a substitution, insertion, deletion and/or fusion, at one or more residue positions, compared to a parent polypeptide. As used herein, the terms “variant Cas12i2 polypeptide” and “variant effector polypeptide” refer to a polypeptide comprising an alteration as compared to the polypeptide of SEQ ID NO: 2. Cas12i2 variants include proteins of SEQ ID NOs: 3-7.

As used herein, the term “deletion” refers to a loss or removal of nucleotides in a nucleic acid sequence relative to a reference sequence. The deletion can be a frameshift mutation or a non-frameshift mutation. A Cas12i-induced deletion described herein refers to a deletion of up to about 100 nucleotides, such as from about 4 nucleotides and 100 nucleotides, from about 4 nucleotides and 50 nucleotides, from about 4 nucleotides and 40 nucleotides, from about 4 nucleotides and 25 nucleotides, from about 10 nucleotides and 25 nucleotides, from about 10 nucleotides and 15 nucleotides, from a nucleic acid molecule. In some embodiments, a Cas12i-induced deletion described herein occurs downstream of a 5′-NTTN-3′ sequence.

As used herein, the term “insertion” refers to a gain of nucleotides in a nucleic acid sequence. The nucleic acid sequence can be in a genome of an organism. The nucleic acid sequence can be in a cell. The nucleic acid sequence can be a DNA sequence. The nucleic acid sequence can be an RNA sequence. The insertion can be a frameshift mutation or a non-frameshift mutation. A Cas12i-induced insertion described herein refers to an insertion of up to about 10 nucleotides. In some embodiments, a Cas12i-induced insertion described herein occurs downstream of a 5′-NTTN-3′ sequence.

As used herein, the term “integration” refers to an insertion of nucleic acids from an exogenous nucleic acid sequence into the genome of a cell. In some embodiments, the integration comprises 10 or more nucleotides (e.g., at least 10, 25, 50, 75, 100, 150, 200, 250, 300, 350, 400, 450, 500, 600, 750, 1000, 1500, 2000, 2500, or 3000 nucleotides). In some embodiments, the integrated nucleic acid comprises a sequence encoding protein, e.g., a chimeric antigen receptor (CAR). In some embodiments, the integrated nucleic acid comprises a sequence encoding a CAR and a regulatory sequence (e.g., a promoter) that directs expression of the CAR in cells (e.g., T cells). In some embodiments, the exogenous nucleic acid is integrated into the genome of a cell by use of homology-directed repair (HDR).

As used herein, the term “protospacer adjacent motif” or “PAM” refers to a DNA sequence adjacent to a target sequence to which a complex comprising a Cas12i polypeptide and an RNA guide binds. In some embodiments, a PAM sequence is required for enzyme activity. In the case of a double-stranded target, the RNA guide binds to a first strand of the target, and a PAM sequence as described herein is present in the second, complementary strand. For example, in some embodiments, the RNA guide binds to the target strand (e.g., the spacer-complementary strand), and the PAM sequence as described herein is present in the non-target strand (i.e., the non-spacer-complementary strand).

As used herein, the term “Cas12i-induced” and the like in reference to a disruption (e.g., a deletion or an insertion) refer to a deletion or an insertion created upon cleavage of a target nucleic acid molecule by a Cas12i polypeptide (e.g., a deletion or insertion directly induced by Cas12i) or a deletion or insertion created following cleavage of a target nucleic acid molecule by Cas12i and DNA repair of the target nucleic acid molecule (e.g., a deletion or insertion indirectly induced by Cas12i). In some embodiments, a Cas12i-induced deletion or insertion is carried out together with an insertion of an exogenous nucleic acid, which optionally encodes a protein (e.g., a CAR) which optionally may also include a regulatory sequence (e.g., a promoter) directing expression of the CAR. In some embodiments, such an insertion of an exogenous nucleic acid occurs at or adjacent to the site of the Cas12i-induced deletion or insertion. In some embodiments, such an insertion of an exogenous nucleic acid occurs distal from the site of the Cas12i-induced deletion or insertion.

As used herein, the term “plurality” indicates “two or more.” As used herein, the term “plurality” in the context of cells refers to two or more cells, such as two or more modified cells. As used herein, the term “plurality of cells” refers to at least about 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,0000, or more cells. In some embodiments, a plurality of cells refers to cells of a cell culture or cell line. In some embodiments, a plurality of cells is referred to as a “population of cells.”

As used herein, the term “progeny” refers to daughter cells resulting from division of one or more parent cells (e.g., modified parent cells). In some embodiments, progeny (e.g., daughter cells) are modified cells. In some embodiments, progeny are daughter cells resulting from one or more modified parent cells. In some embodiments, progeny are multi-generational, e.g., daughter cells of modified parent cells can be used to generate further daughter cells, and so on. It will be understood that more than one generation of progeny are envisioned.

As used herein, the term “reference sequence” refers to an unmodified nucleic acid sequence. The reference sequence can be a nucleic acid sequence not modified by a Cas12i polypeptide (e.g., a deletion or insertion directly induced by Cas12i). The reference sequence can be an unmodified genome of an organism. The reference sequence can be an unmodified genome of an organism. The reference sequence can be an unmodified nucleic acid sequence in a cell. The reference sequence can be an unmodified DNA sequence. The nucleic acid sequence can be an unmodified RNA sequence.

As used herein, the term “substantial” refers to a measurable, considerable, or ample amount. In some embodiments, the term “substantial” is used to refer to the expression level of a gene. In some embodiments wherein a modified cell lacks substantial expression of a gene, expression of the gene in the modified cell is 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, or any integer therebetween) the expression of the gene in an unmodified cell. In some embodiments wherein a modified cell lacks substantial expression of a gene, the modified cell does not express the gene.

As used herein, the terms “upstream” and “downstream” refer to relative positions within a single nucleic acid (e.g., DNA) sequence in a nucleic acid molecule. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence. In some embodiments, the 5′-NTTN-3′ sequence is upstream of an insertion or deletion described herein, and the insertion or deletion is downstream of the 5′-NTTN-3′ sequence. In some embodiments, “downstream” in reference to a deletion or insertion refers to the relative position in the non-target strand (i.e., the non-spacer-complementary strand). In embodiments wherein a deletion or insertion is downstream of a 5′-NTTN-3′ sequence of the non-target strand, the deletion or insertion can also be described as being upstream of a 5′-NAAN-3′ sequence on the target strand (i.e., the spacer complementary strand). In embodiments wherein a deletion or insertion is downstream of a 5′-NTTN-3′ sequence of the sense strand (e.g., coding strand), the deletion or insertion can also be described as being upstream of a 5′-NAAN-3′ sequence on the antisense strand (e.g., non-coding strand). In embodiments wherein a deletion or insertion is downstream of a 5′-NTTN-3′ sequence of the antisense strand (e.g., non-coding strand), the deletion or insertion can also be described as being upstream of a 5′-NAAN-3′ sequence on the sense strand (e.g., coding strand).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A is a schematic showing site-specific HDR knock-in of donor dsDNA encoding a CAR into the TRAC locus of a T cell using Cas12i RNP.

FIG. 1B is a schematic showing the use of a CAR T cell generated as per the method illustrated in FIG. 1 in the killing of cancer cells.

FIG. 2A is a schematic of a representative adeno-associated virus (AAV) vector that includes cDNA encoding a CAR and homology arms flanking the cut site into which CAR encoding sequences are to be inserted.

FIG. 2B is a schematic of the sequence of a CAR directed towards CD19.

### DETAILED DESCRIPTION

The present disclosure relates to a modified cell comprising a DNA deletion and/or DNA insertion induced by a Cas12i nuclease. In some aspects, the deletion and/or insertion is a disruption of a gene (e.g., a gene encoding TRAC or B2M). In some aspects, a modified cell having one or more characteristics is described herein. In some aspects, the modified cell is a T cell. In some aspects, the modified cell comprises an insertion of an exogenous nucleic acid, e.g., a nucleic acid encoding a protein (e.g., a chimeric antigen receptor (CAR)) to be expressed in the cell. In some aspects, the modified cell is a T cell and comprises an insertion of a sequence encoding a chimeric antigen receptor (CAR). In some aspects, the modified cell comprises a DNA integration from a template DNA as described in section “Production” induced by a Cas12i nuclease described herein. In some aspects, a method of producing the modified cell is described. In some aspects, a composition or formulation comprises the modified cell described herein or a plurality of the modified cells described herein.

### Modified Cells

In some aspects, the disclosure described herein comprises a modified cell or a plurality of modified cells (e.g., a modified T cell or a plurality of modified T cells). In some embodiments, the modified cell is a genetically modified cell. In some embodiments, the modified cell is a cell comprising a disruption in its genome, e.g., a deletion and/or an insertion. In some embodiments, the modified cell is a cell comprising an insertion of an exogenous nucleic acid. In some embodiments, the modified cell comprises a biochemical modification.

### Cell Type

The modified cell or plurality of modified cells described herein can be a variety of cells. In some embodiments, the cell is an isolated cell. In some embodiments, the cell is in cell culture or a co-culture of two or more cell types. In some embodiments, the cell is ex vivo. In some embodiments, the cell is obtained from a living organism and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is a mammalian cell, e.g., a human cell or a murine cell. In some embodiments, the murine cell is derived from a wild-type mouse, an immunosuppressed mouse, or a disease-specific mouse model. In some embodiments, the cell is a cell within a living tissue, organ, or organism.

In some embodiments, the cell is a T cell. In some embodiments, the T cell is a T helper cell (i.e., a CD4+ T cell). In some embodiments, the T cell is a T helper cell selected from Th1, Th2, Th17, Th9, or Tfh cells. In some embodiments, the T cell is a cytotoxic T cell (i.e., a CD8+ T cell). In some embodiments, the T cell is a CD4+/CD8+ T cell. In some embodiments, the T cell is an effector T cell. In some embodiments, the T cell is a memory T cell (e.g., a central memory T cell, an effector memory T cell, a tissue resident T cell, or a virtual memory T cell). In some embodiments, the T cell is a regulatory T cell (e.g., a FOXP3+Treg or a FOXP3-Treg cell). In some embodiments, the T cell is activated. In some embodiments, the T cell is naïve. In some embodiments, the T cell is an innate-like T cell. In some embodiments, the T cell is a natural killer T cell, a mucosal associated invariant T cell, or a gamma delta T cell. In some embodiments, the T cell is obtained from a sample of a subject to whom it (or progeny thereof) is to be administered after genetic modification. In some embodiments, the T cell is obtained from a different subject from that to whom it (or progeny thereof) is to be administered after genetic modification. In some embodiments, the sample comprises peripheral blood mononuclear cells (PBMCs).

### Genetic Characteristics

In some embodiments, the modified cell (e.g., a T cell) comprises a modification in a genomic region or a gene. In some embodiments, the gene is a TRAC gene. In some embodiments, the gene is a B2M gene. In some embodiments, the modification is in an exon region of a gene. In some embodiments, the modification is in an intron region of a gene. In some embodiments, the modification is in a promoter region of a gene. In some embodiments, the modification is in an enhancer region of a gene. In some embodiments, the modification is in a silencer region of a gene. In some embodiments, the modification is in a terminator region of a gene. In some embodiments, the modification is in a region that regulates transcription of a gene. In some embodiments, the modification results in an altered expression (e.g., increase or decrease) of a gene product.

In some embodiments, the modified cell comprises two or more modifications (e.g., two or more desired or targeted modifications). In some embodiments, the modified cell comprises two or more deletions in the same gene. In some embodiments, the modified cell comprises a deletion in a first gene and a deletion in a second gene. In some embodiments, the modified cell comprises two or more insertions in the same gene. In some embodiments, the modified cell comprises an insertion in a first gene and an insertion in a second gene. In some embodiments, the modified cell comprises two or more indels (e.g., at least one deletion and at least one insertion) in the same gene. In some embodiments, the modified cell comprises an indel (e.g., deletion or insertion) in a first gene and an indel (e.g., deletion or insertion) in a second gene. In some embodiments, the modified cell comprises an insertion of an exogenous nucleic acid (e.g., an exogenous nucleic acid comprising a sequence encoding a protein, such as a CAR, and optionally regulatory sequences controlling expression of the protein encoding sequence). In some embodiments, the insertion of the exogenous nucleic acid is at the site of a Cas12i-induced deletion or insertion as described herein. In some embodiments, the insertion of the exogenous nucleic acid is at a site that is distal from the site of a Cas12i-induced deletion or insertion.

In some embodiments, the gene having the modification is present in the nucleus of a cell as described elsewhere herein. In some embodiments, the gene having the modification is endogenous to the cell. In some embodiments, the gene having the modification is a genomic DNA. In some embodiments, the gene having the modification is a chromosomal DNA. In some embodiments, the gene having the modification is a protein-coding gene or a functional region thereof, such as a coding region, or a regulatory element, such as a promoter, enhancer, a 5′ or 3′ untranslated region, etc. In some embodiments, the modification is in an exon or an intron. In some embodiments, the gene having the modification is a non-coding gene, such as transposon, miRNA, tRNA, ribosomal RNA, ribozyme, or lncRNA.

In some embodiments, the modification alters expression of the gene. In some embodiments, the modification alters function of the gene. In some embodiments, the modification inactivates the gene. In some embodiments, the modification is a frameshifting modification. In some embodiments, the modification is a non-frameshifting modification. In some embodiments, the modification leads to cell toxicity or cell death (e.g., apoptosis).

In some embodiments, the modified cell comprises one or more insertions and/or deletions (e.g., disruptions) described herein. In some embodiments, the modification (e.g., deletion or insertion) overlaps with a mutation in the gene. In some embodiments, the modification (e.g., deletion) overlaps with an insertion within the gene. For example, in some embodiments, the modification (e.g., deletion) removes at least a portion of a repeat expansion of the gene. In some embodiments, the modification (e.g., insertion) overlaps with a deletion within the gene. In some embodiments, the modification (e.g., insertion) corrects a deletion in a gene. In some embodiments, the insertion or deletion corrects a frameshift in a gene. In some embodiments, the modification disrupts one allele of the gene. In some embodiments, the modification disrupts both alleles of the gene. In some embodiments, the disruption is an insertion, deletion and/or substitution in the genome of a cell.

In some embodiments, the modified cell comprises a deletion or insertion (e.g., one or more deletions and/or one or more insertions) described herein and further comprises an exogenous nucleic acid integrated into the genome of the cell.

In some embodiments, the modified cell (e.g., T cell) comprises an insertion (i.e., integration) of an exogenous nucleic acid. In some embodiments the exogenous nucleic acid is inserted (i.e., integrated) into a gene of the modified cell. In some embodiments the exogenous nucleic acid is integrated into a gene of the modified cell. In some embodiments, the exogenous nucleic acid comprises a sequence encoding a protein, e.g., a CAR, and optionally a regulatory sequence controlling expression of the sequence encoding the protein. In some embodiments the exogenous nucleic acid sequence is flanked by homology arms that assist in targeted integration of the nucleic acid sequence into the genome of the modified T cell. In some embodiments, the insertion is at least about 10 nucleotides (e.g., at least about 25, 50, 75, 100, 150, 200, 250, 300, 350, 400, 450, 500, 600, 750, 1000, 1500, 2000, 2500, or 3000 nucleotides) in length. In some embodiments, the insertion is at least a 10-nucleotide insertion. In some embodiments, the insertion is at least a 50-nucleotide insertion. In some embodiments, the insertion is at least a 100-nucleotide insertion. In some embodiments, the insertion is at least a 500-nucleotide insertion. In some embodiments, the insertion is at least a 750-nucleotide insertion. In some embodiments, the insertion is at least a 1000-nucleotide insertion. In some embodiments, the insertion is at least a 1500-nucleotide insertion. In some embodiments, the insertion is at least a 2000-nucleotide insertion. In some embodiments, the insertion is at least a 2500-nucleotide insertion. In some embodiments, the insertion is longer than 3000 nucleotides.

In some embodiments, the insertion of the exogenous nucleic acid is in a gene that is expressed in T cells. In some embodiments, the insertion of the exogenous nucleic acid in a gene knocks out expression of the gene. In some embodiments, the insertion of the exogenous nucleic acid in the gene prevents expression of a functional gene product in the cell. In some embodiments, the insertion of the exogenous nucleic acid in the gene reduces mRNA expression in the cell. In some embodiments, the insertion of the exogenous nucleic acid in the gene reduces protein levels in the cell.

In some embodiments, the gene expression profile of a modified cell described herein is altered, as compared to an unmodified cell. In some embodiments, expression of the disrupted gene is decreased by about 0.5%, 1%, 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or any integer therebetween) as compared to expression of a reference gene (e.g., an unmodified gene in an unmodified cell). In some embodiments, expression of the disrupted gene is 95%, 90%, 85%, 80%, 75%, 70%, 65%, 60%, 55%, 50%, 45%, 40%, 35%, 30%, 25%, 20%, 15%, 10%, or 5% the expression of a reference gene (e.g., an unmodified gene in an unmodified cell). In some embodiments, expression of the disrupted gene is increased by at least about 0.5%, 1%, 5%, 10%, 15%, 20%, 25%, 30%, 35%, 40%, 45%, 50%, 55%, 60%, 65%, 70%, 75%, 80%, 85%, 90%, 95%, 100%, 200%, or 300%, as compared to expression of a reference gene (e.g., an unmodified gene in an unmodified cell).

In some embodiments, the modified gene is an immune-related gene, for example, a gene that is involved in an immune response in a subject. In some embodiments, the modified gene is an immune checkpoint gene. In some embodiments, the modified gene is selected from the group consisting of: BCL11A intronic erythroid enhancer, CD3, Beta-2 microglobulin (B2M), T Cell Receptor Alpha Constant (TRAC), Programmed Cell Death 1 (PDCD1), T-cell receptor alpha, T-cell receptor beta, B-cell lymphoma/leukemia 11A (BCL11A), Cytotoxic T-Lymphocyte Antigen 4 (CTLA-4), chemokine (C-C motif) receptor 5 (gene/pseudogene) (CCR5), CXCR4 gene, CD160 molecule (CD160), adenosine A2a receptor (ADORA), CD276, B7-H3, B7-H4, BTLA, nicotinamide adenine dinucleotide phosphate NADPH oxidase isoform 2 (NOX2), V-domain Ig suppressor of T cell activation (VISTA), Sialic acid-binding immunoglobulin-type lectin 7 (SIGLEC7), Sialic acid-binding immunoglobulin-type lectin 9 (SIGLEC9), SIGLEC10, V-set domain containing T cell activation inhibitor 1 (VTCN1), B and T lymphocyte associated (BTLA), Indoleamine 2,3-dioxygenase (IDO), indoleamine 2,3-dioxygenase 1 (IDO1), Killer-cell Immunoglobulin-like Receptor (KIR), killer cell immunoglobulin-like receptor, three domains, long cytoplasmic tail, 1 (KIR3DL1), lymphocyte-activation gene 3 (LAG3), T-cell Immunoglobulin domain and Mucin domain 3 (TIM3), hepatitis A virus cellular receptor 2 (HAVCR2), natural killer cell receptor 2B4 (CD244), hypoxanthine phosphoribosyltransferase 1 (HPRT), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIR1), adeno-associated virus integration site 1 (AAVS1), AAVS 2, AAVS3, AAVS4, AAVS5, AAVS6, AAVS7, AAVS8, transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), TGFB induced factor homeobox 1 (TGIF1), tumor necrosis factor receptor superfamily member, tumor necrosis factor receptor superfamily member 10b (TNFRSF10B), tumor necrosis factor receptor superfamily member 10a (TNFRSF10A), BY55, B7H5, caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), guanylate cyclase 1, soluble, beta 3 (GUCY1B3), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, CD27, CD28, CD40, CD122, CD137, OX40, GITR, and ICOS. In some embodiments, the modified gene is Programmed Death Ligand 1 (PD-L1), Class II Major Histocompatibility Complex Transactivator (CIITA), Adeno-Associated Virus Integration Site 1 (AAVS1), Citramalyl-CoA lyase (CLYBL), Transthyretin (TTR), Lactate Dehydrogenase-A (LDHA), Hydroxyacid Oxidase-1 (HAO1), Alanine-Glyoxylate and Serine-Pyruvate Aminotransferase (AGXT), Glyoxylate Reductase/Hydroxypyruvate Reductase (GRHPR), or 4-Hydroxy-2-Oxoglutarate Aldolase (HOGA).

In some embodiments, the modified cell is a modified T cell and comprises an insertion of a sequence encoding a CAR (hereinafter a “CAR insertion”), which also optionally includes a regulatory sequence directing expression of the CAR, upstream of a 5′-NTTN-3′ sequence. In some embodiments, the modified T cell comprises the CAR insertion upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR′3′, 5′-CTTR-3′, 5′-DTTT-3′, 5′-ATTN-3′, or 5′-GTTN-3′ sequence. In some embodiments, the modified T cell comprises the CAR insertion upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the modified T cell comprises the CAR insertion upstream of a T/C-rich sequence.

In some embodiments, the insertion (e.g., a CAR) is in a gene that is expressed in T cells. In some embodiments, the insertion (e.g., CAR) in the gene knocks out expression of the gene. In some embodiments, the insertion (e.g., CAR) in the gene prevents expression of a functional gene product from the gene. In some embodiments, the insertion is in a gene encoding T-cell receptor alpha constant region (TRAC) or beta-2 microglobulin (B2M). In some embodiments, a cell comprises an insertion (e.g., a CAR) in a gene encoding TRAC, and the cell further comprises an indel in a gene encoding B2M. In some embodiments, a cell comprises an insertion (e.g., a CAR) in a gene encoding TRAC, and the cell further does not express TRAC or B2M. In some embodiments, the insertion (e.g., a CAR) is in exon 1 of a gene encoding TRAC. In some embodiments, the insertion (e.g., a CAR) is in exon 2 of a gene encoding TRAC.

In some embodiments, the insertion (e.g., a CAR) is in exon 3 of a gene encoding TRAC. In some embodiments, the insertion (e.g., a CAR) is in exon 4 of a gene encoding TRAC. In preferred embodiments, the insertion is towards the beginning of the coding sequence to minimize TRAC sequence expression. In some embodiments, the gene encoding B2M has the sequence of SEQ ID NO: 8. In some embodiments, the insertion (e.g., a CAR) is in exon 1 of a gene encoding B2M. In some embodiments, the insertion (e.g., a CAR) is in exon 2 of a gene encoding B2M. In some embodiments, the insertion (e.g., a CAR) is in exon 3 of a gene encoding B2M. In some embodiments, the insertion (e.g., a CAR) is in exon 4 of a gene encoding B2M. In preferred embodiments, the insertion is towards the beginning of the coding sequence to minimize B2M sequence expression. In some embodiments, the gene encoding B2M has the sequence of SEQ ID NO: 9.

In some embodiments, the CAR insertion starts within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) of the 5′-NTTN-3′ sequence. In some embodiments, the CAR insertion starts within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) of a T/C-rich sequence.

In some embodiments, the CAR insertion starts within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) downstream of the 5′-NTTN-3′ sequence. In some embodiments, the CAR insertion starts within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) downstream of a T/C-rich sequence.

In some embodiments, the CAR insertion starts within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) upstream of the 5′-NTTN-3′ sequence. In some embodiments, the CAR insertion starts within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) upstream of a T/C-rich sequence.

In some embodiments, the CAR insertion starts within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) of the 5′-NTTN-3′ sequence. In some embodiments, the CAR insertion starts within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) of a T/C-rich sequence.

In some embodiments, the CAR insertion starts within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of the 5′-NTTN-3′ sequence. In some embodiments, the CAR insertion starts within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a T/C-rich sequence.

In some embodiments, the CAR insertion starts within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) upstream of the 5′-NTTN-3′ sequence. In some embodiments, the CAR insertion starts within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) upstream of a T/C-rich sequence.

In some embodiments, the CAR insertion starts within about 3 nucleotides (e.g., 1, 2 or 3 nucleotides) upstream to about 35 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of the 5′-NTTN-3′ sequence. In some embodiments, the CAR insertion starts within about 3 nucleotides (e.g., 1, 2 or 3 nucleotides) upstream to about 35 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 3 nucleotides (e.g., 1, 2, or 3 nucleotides) upstream to about 35 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 3 nucleotides (e.g., 1, 2, or 3 nucleotides) upstream to about 35 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a T/C-rich sequence.

In some embodiments, the CAR insertion starts within about 3 to about 15 nucleotides (e.g., about 3, 4, 5, 6, 7, 8, 9, 10, 11, 12, 13, 14, or 15 nucleotides) of the 5′-NTTN-3′ sequence. In some embodiments, the CAR insertion starts within about 3 to about 15 nucleotides (e.g., about 3, 4, 5, 6, 7, 8, 9, 10, 11, 12, 13, 14, or 15 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 3 to about 15 nucleotides (e.g., about 3, 4, 5, 6, 7, 8, 9, 10, 11, 12, 13, 14, or 15 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 3 to about 15 nucleotides (e.g., about 3, 4, 5, 6, 7, 8, 9, 10, 11, 12, 13, 14, or 15 nucleotides) of a T/C-rich sequence.

In some embodiments, the CAR insertion starts within about 3 to about 15 nucleotides (e.g., about 3, 4, 5, 6, 7, 8, 9, 10, 11, 12, 13, 14, or 15 nucleotides) downstream of the 5′-NTTN-3′ sequence. In some embodiments, the CAR insertion starts within about 3 to about 15 nucleotides (e.g., about 3, 4, 5, 6, 7, 8, 9, 10, 11, 12, 13, 14, or 15 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 3 to about 15 nucleotides (e.g., about 3, 4, 5, 6, 7, 8, 9, 10, 11, 12, 13, 14, or 15 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 3 to about 15 nucleotides (e.g., about 3, 4, 5, 6, 7, 8, 9, 10, 11, 12, 13, 14, or 15 nucleotides) downstream of a T/C-rich sequence.

In some embodiments, the CAR insertion starts within about 3 to about 15 nucleotides (e.g., about 3, 4, 5, 6, 7, 8, 9, 10, 11, 12, 13, 14, or 15 nucleotides) upstream of the 5′-NTTN-3′ sequence. In some embodiments, the CAR insertion starts within about 3 to about 15 nucleotides (e.g., about 3, 4, 5, 6, 7, 8, 9, 10, 11, 12, 13, 14, or 15 nucleotides) upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 3 to about 15 nucleotides (e.g., about 3, 4, 5, 6, 7, 8, 9, 10, 11, 12, 13, 14, or 15 nucleotides) upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 3 to about 15 nucleotides (e.g., about 3, 4, 5, 6, 7, 8, 9, 10, 11, 12, 13, 14, or 15 nucleotides) upstream of a T/C-rich sequence.

In some embodiments, the CAR insertion starts within about 15 to about 30 nucleotides (e.g., about 15, 16, 17, 18, 19, 20, 21, 22, 23, 24, 25, 26, 27, 28, 29, or 30 nucleotides) of the 5′-NTTN-3′ sequence. In some embodiments, the CAR insertion starts within about 15 to about 30 nucleotides (e.g., about 15, 16, 17, 18, 19, 20, 21, 22, 23, 24, 25, 26, 27, 28, 29, or 30 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 15 to about 30 nucleotides (e.g., about 15, 16, 17, 18, 19, 20, 21, 22, 23, 24, 25, 26, 27, 28, 29, or 30 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 15 to about 30 nucleotides (e.g., about 15, 16, 17, 18, 19, 20, 21, 22, 23, 24, 25, 26, 27, 28, 29, or 30 nucleotides) of a T/C-rich sequence.

In some embodiments, the CAR insertion starts within about 15 to about 30 nucleotides (e.g., about 15, 16, 17, 18, 19, 20, 21, 22, 23, 24, 25, 26, 27, 28, 29, or 30 nucleotides) downstream of the 5′-NTTN-3′ sequence. In some embodiments, the CAR insertion starts within about 15 to about 30 nucleotides (e.g., about 15, 16, 17, 18, 19, 20, 21, 22, 23, 24, 25, 26, 27, 28, 29, or 30 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 15 to about 30 nucleotides (e.g., about 15, 16, 17, 18, 19, 20, 21, 22, 23, 24, 25, 26, 27, 28, 29, or 30 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 15 to about 30 nucleotides (e.g., about 15, 16, 17, 18, 19, 20, 21, 22, 23, 24, 25, 26, 27, 28, 29, or 30 nucleotides) downstream of a T/C-rich sequence.

In some embodiments, the CAR insertion starts within about 15 to about 30 nucleotides (e.g., about 15, 16, 17, 18, 19, 20, 21, 22, 23, 24, 25, 26, 27, 28, 29, or 30 nucleotides) upstream of the 5′-NTTN-3′ sequence. In some embodiments, the CAR insertion starts within about 15 to about 30 nucleotides (e.g., about 15, 16, 17, 18, 19, 20, 21, 22, 23, 24, 25, 26, 27, 28, 29, or 30 nucleotides) upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the CAR insertion starts within about 15 to about 30 nucleotides (e.g., about 15, 16, 17, 18, 19, 20, 21, 22, 23, 24, 25, 26, 27, 28, 29, or 30 nucleotides) upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the CAR insertion starts within about 15 to about 30 nucleotides (e.g., about 15, 16, 17, 18, 19, 20, 21, 22, 23, 24, 25, 26, 27, 28, 29, or 30 nucleotides) upstream of a T/C-rich sequence.

In some embodiments, the CAR insertion is at least about 1000 nucleotides (e.g., at least about 1000, 1500, 2000, 2500, or 3000 nucleotides) in length. In some embodiments, the CAR insertion is at least a 10-nucleotide insertion. In some embodiments, the CAR insertion is at least a 50-nucleotide insertion. In some embodiments, the CAR insertion is at least a 100-nucleotide insertion. In some embodiments, the CAR insertion is at least a 500-nucleotide insertion. In some embodiments, the CAR insertion is at least a 750-nucleotide insertion. In some embodiments, the CAR insertion is at least a 1000-nucleotide insertion. In some embodiments, the CAR insertion is at least a 1500-nucleotide insertion. In some embodiments, the CAR insertion is at least a 2000-nucleotide insertion. In some embodiments, the CAR insertion is at least a 2500-nucleotide insertion. In some embodiments, the insertion is longer than 3000 nucleotides.

In some embodiments, a modified cell comprises a disruption (e.g., deletion and/or insertion) in one or more genes in a cell. In some embodiments, a modified cell comprises a disruption in a TRAC gene and/or a B2M gene. In some embodiments, a modified cell comprises an exogenous nucleic acid (e.g., a CAR) integrated into a TRAC gene and/or a B2M gene. In some embodiments, a modified cell comprises a disruption in a TRAC gene and an exogenous nucleic acid (e.g., a CAR) integrated into the B2M gene. In some embodiments, a modified cell comprises a disruption in a B2M gene and an exogenous nucleic acid (e.g., a CAR) integrated into a TRAC gene.

### Biochemical Characteristics

In some embodiments, a modified cell (e.g., modified T cell) described herein is further characterized by a biochemical change, as compared to a non-modified cell. In some embodiments, the biochemical change that occurs is transient. For example, in some embodiments, the biochemical change occurs while the cell is being modified or after the cell has been modified.

In some embodiments, the biochemical change occurs at the initiation of DNA repair, during DNA repair, or after DNA repair. In some embodiments, the modified cell of the disclosure is characterized by a stimulated cellular endogenous DNA repair pathway. In some embodiments, the stimulated DNA repair pathway is Non-Homologous End Joining (NHEJ), Alternative Non-Homologues End-Joining (A-NHEJ), or Homology Directed Recombination (HDR). NHEJ can repair cleaved target sequence without the need for a homologous template. NHEJ can result in an insertion as described herein. In some embodiments, NHEJ results in insertion of one or more nucleotides at the target sequence. HDR can occur with a homologous template, such as the donor DNA. In some embodiments, the homologous template can comprise sequences that are homologous to sequences flanking the target sequence cleavage site. The homologous sequences are referred to as homology arms and flank an exogenous nucleotide sequence to be inserted into the target sequence. In some cases, HDR can insert an exogenous nucleotide sequence into the cleaved target sequence. The modifications of the target DNA due to NHEJ and/or HDR can further lead to, for example, mutations, insertions, alterations, integrations, gene correction, gene replacement, gene tagging, transgene knock-in, gene disruption, and/or gene knock-outs.

In some embodiments, the modified cell (e.g., modified T cell) of the disclosure is characterized by recruitment of one or more endogenous cellular molecules. In some embodiments, the modified cell is characterized by recruitment of one or more molecules not involved in a DNA repair pathway. In some embodiments, the one or more recruited molecules associate with the genomic DNA of the cell. In some embodiments, one or more signal transduction pathways of a modified cell differ from those of a non-modified cell. For example, in some embodiments, the modified cell is characterized by a release of one or more secondary messengers. In some embodiments, the gene expression profile of a modified cell described herein is altered, as compared to a non-modified cell.

### Plurality of Cells

In some embodiments, a cell of a plurality of cells (e.g., T cells) comprises at least one deletion and/or at least one insertion. In some embodiments, the deletion and/or insertion is in a gene. In some embodiments the gene encodes TRAC. In some embodiments, the gene encodes B2M. In some embodiments, a cell of the plurality of cells comprises an insertion of an exogenous nucleic acid. In some embodiments, the exogenous nucleic acid comprises a sequence encoding a protein (e.g., a CAR) and optionally a regulatory sequence that controls expression of the sequence encoding a protein. In some embodiments, the plurality of cells comprises a disruption (e.g., deletion and/or insertion) in one or more genes in a cell. In some embodiments, the plurality of cells comprises an insertion of an exogenous nucleic acid as described herein.

In some embodiments, a plurality of modified cells comprises a disruption in a TRAC gene and/or a B2M gene. In some embodiments, a plurality of modified cells comprises a disruption in a TRAC gene and an exogenous nucleic acid (e.g., a CAR) integrated into the B2M gene. In some embodiments, a plurality of modified cells comprises a disruption in a B2M gene and an exogenous nucleic acid (e.g., a CAR) integrated into a TRAC gene. Details of Cas12i-induced disruptions (e.g., insertions and deletions) are provided below in the Production section.

In some embodiments, a plurality of cells is obtained by culturing a modified cell (e.g., a modified T cell) comprising an insertion described herein. In some embodiments, a plurality of cells is obtained by isolating and culturing a modified cell comprising an insertion described herein. In some embodiments, a plurality of cells is obtained by culturing one or more cells comprising an insertion. In some embodiments, a plurality of cells is obtained by culturing one or more modified cells.

In some embodiments, the cells of the plurality of cells are T cells and a T cell of the plurality of T cells comprises an insertion of a sequence encoding a protein, such as a CAR, which also optionally includes a regulatory sequence directing expression of the protein, e.g., the CAR. In some embodiments, two or more T cells of a plurality of T cells comprise a CAR insertion. In some embodiments, the CAR insertion is at least about 1000 nucleotides (e.g., at least about 1000, 1500, 2000, 2500, or 3000 nucleotides) in length.

In some embodiments, a plurality of T cells is obtained by culturing a modified T cell comprising a CAR insertion described herein. In some embodiments, a plurality of T cells is obtained by isolating and culturing a modified T cell comprising a CAR insertion described herein. In some embodiments, a plurality of T cells is obtained by culturing one or more T cells comprising a CAR insertion. In some embodiments, a plurality of T cells is obtained by culturing one or more modified T cells.

In some embodiments, at least 10% of the T cells of the plurality (e.g., at least about 10%, 15%, 20%, 25%, 30%, 35%, 40%, 45%, 50%, 55%, 60%, 65%, 70%, 75%, 80%, 85%, 90%, 95%, or 100% of the T cells) comprise a CAR insertion in a gene. In some embodiments, at least 20% of the T cells of the plurality (e.g., at least about 20%, 25%, 30%, 35%, 40%, 45%, 50%, 55%, 60%, 65%, 70%, 75%, 80%, 85%, 90%, 95%, or 100% of the T cells) comprise a CAR insertion in a gene. In some embodiments, at least 30% of the T cells of the plurality (e.g., at least about 30%, 35%, 40%, 45%, 50%, 55%, 60%, 65%, 70%, 75%, 80%, 85%, 90%, 95%, or 100% of the T cells) comprise a CAR insertion in a gene. In some embodiments, at least 40% of the T cells of the plurality (e.g., at least about 40%, 45%, 50%, 55%, 60%, 65%, 70%, 75%, 80%, 85%, 90%, 95%, or 100% of the T cells) comprise a CAR insertion in a gene. In some embodiments, at least 50% of the T cells of the plurality (e.g., at least about 50%, 55%, 60%, 65%, 70%, 75%, 80%, 85%, 90%, 95%, or 100% of the T cells) comprise a CAR insertion in a gene. In some embodiments, at least 60% of the T cells of the plurality (e.g., at least about 60%, 65%, 70%, 75%, 80%, 85%, 90%, 95%, or 100% of the T cells) comprise a CAR insertion in a gene. In some embodiments, at least 70% of the T cells of the plurality (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the T cells) comprise a CAR insertion in a gene. In some embodiments, at least 80% of the T cells of the plurality (e.g., at least about 80%, 81%, 82%, 83%, 84%, 85%, 86%, 87%, 88%, 89%, 90%, 91%, 92%, 93%, 94%, 95%, 96%, 97%, 98%, 99%, or 100% of the T cells) comprise a CAR insertion in a gene. In some embodiments, at least 90% of the T cells of the plurality (e.g., at least about 90%, 91%, 92%, 93%, 94%, 95%, 96%, 97%, 98%, 99%, or 100% of the T cells) comprise a CAR insertion in a gene. In some embodiments, each of the T cells of the plurality (e.g., 100% of the T cells) comprises a CAR insertion in a gene.

In some embodiments, at least 10% of the T cells of the plurality (e.g., at least about 10%, 15%, 20%, 25%, 30%, 35%, 40%, 45%, 50%, 55%, 60%, 65%, 70%, 75%, 80%, 85%, 90%, 95%, or 100% of the T cells) comprise a CAR insertion in the same gene. In some embodiments, at least 20% of the T cells of the plurality (e.g., at least about 20%, 25%, 30%, 35%, 40%, 45%, 50%, 55%, 60%, 65%, 70%, 75%, 80%, 85%, 90%, 95%, or 100% of the T cells) comprise a CAR insertion in the same gene. In some embodiments, at least 30% of the T cells of the plurality (e.g., at least about 30%, 35%, 40%, 45%, 50%, 55%, 60%, 65%, 70%, 75%, 80%, 85%, 90%, 95%, or 100% of the T cells) comprise a CAR insertion in the same gene. In some embodiments, at least 40% of the T cells of the plurality (e.g., at least about 40%, 45%, 50%, 55%, 60%, 65%, 70%, 75%, 80%, 85%, 90%, 95%, or 100% of the T cells) comprise a CAR insertion in the same gene. In some embodiments, at least 50% of the T cells of the plurality (e.g., at least about 50%, 55%, 60%, 65%, 70%, 75%, 80%, 85%, 90%, 95%, or 100% of the T cells) comprise a CAR insertion in the same gene. In some embodiments, at least 60% of the T cells of the plurality (e.g., at least about 60%, 65%, 70%, 75%, 80%, 85%, 90%, 95%, or 100% of the T cells) comprise a CAR insertion in the same gene. In some embodiments, at least 70% of the T cells of the plurality (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the T cells) comprise a CAR insertion in the same gene. In some embodiments, at least 80% of the T cells of the plurality (e.g., at least about 80%, 81%, 82%, 83%, 84%, 85%, 86%, 87%, 88%, 89%, 90%, 91%, 92%, 93%, 94%, 95%, 96%, 97%, 98%, 99%, or 100% of the T cells) comprise a CAR insertion in the same gene. In some embodiments, at least 90% of the T cells of the plurality (e.g., at least about 90%, 91%, 92%, 93%, 94%, 95%, 96%, 97%, 98%, 99%, or 100% of the T cells) comprise a CAR insertion in the same gene. In some embodiments, each of the T cells of the plurality (e.g., 100% of the T cells) comprises a CAR insertion in the same gene.

In some embodiments, two or more cells of a plurality of T cells comprise an identical CAR insertion (e.g., the same CAR insertion). In some embodiments, at least 10% of the T cells of the plurality (e.g., at least about 10%, 15%, 20%, 25%, 30%, 35%, 40%, 45%, 50%, 55%, 60%, 65%, 70%, 75%, 80%, 85%, 90%, 95%, or 100% of the T cells) comprise the same CAR insertion. In some embodiments, at least 20% of the T cells of the plurality (e.g., at least about 20%, 25%, 30%, 35%, 40%, 45%, 50%, 55%, 60%, 65%, 70%, 75%, 80%, 85%, 90%, 95%, or 100% of the T cells) comprise the same CAR insertion. In some embodiments, at least 30% of the T cells of the plurality (e.g., at least about 30%, 35%, 40%, 45%, 50%, 55%, 60%, 65%, 70%, 75%, 80%, 85%, 90%, 95%, or 100% of the T cells) comprise the same CAR insertion. In some embodiments, at least 40% of the T cells of the plurality (e.g., at least about 40%, 45%, 50%, 55%, 60%, 65%, 70%, 75%, 80%, 85%, 90%, 95%, or 100% of the T cells) comprise the same CAR insertion. In some embodiments, at least 50% of the T cells of the plurality (e.g., at least about 50%, 55%, 60%, 65%, 70%, 75%, 80%, 85%, 90%, 95%, or 100% of the T cells) comprise the same CAR insertion. In some embodiments, at least 60% of the T cells of the plurality (e.g., at least about 60%, 65%, 70%, 75%, 80%, 85%, 90%, 95%, or 100% of the T cells) comprise the same CAR insertion. In some embodiments, at least 70% of the T cells of the plurality (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the T cells) comprise the same CAR insertion. In some embodiments, at least 80% of the T cells of the plurality (e.g., at least about 80%, 81%, 82%, 83%, 84%, 85%, 86%, 87%, 88%, 89%, 90%, 91%, 92%, 93%, 94%, 95%, 96%, 97%, 98%, 99%, or 100% of the T cells) comprise the same CAR insertion. In some embodiments, at least 90% of the T cells of the plurality (e.g., at least about 90%, 91%, 92%, 93%, 94%, 95%, 96%, 97%, 98%, 99%, or 100% of the T cells) comprise the same CAR insertion. In some embodiments, each of the T cells of the plurality (e.g., 100% of the T cells) comprises the same CAR insertion.

### Production

The disclosure also provides methods of obtaining a modified cell (e.g., a modified T cell) of the disclosure. In some embodiments, the methods comprise introducing (a) a Cas12i polypeptide (e.g., a variant Cas12i polypeptide) or a nucleic acid encoding the Cas12i polypeptide (e.g., a variant Cas12i polypeptide), (b) a guide nucleic acid (e.g., RNA guide) or a nucleic acid encoding the guide nucleic acid (e.g., RNA guide), and (c) an exogenous nucleic acid into a cell (e.g., a T cell), e.g., for integration into the genome of the cell. In some embodiments, the methods comprise (a) disrupting a gene in the genome of a T cell using a variant Cas12i polypeptide and a guide nucleic acid (e.g., an RNA guide), and (b) introducing an exogenous nucleic acid into the genome of the T cell. In some embodiments, the methods comprise introducing into a T cell: (a) a variant Cas12i polypeptide, or a nucleic acid encoding the variant Cas12i polypeptide, and a first RNA guide, or a nucleic acid encoding the first RNA guide, for inducing a deletion or an insertion in the genome of the T cell; and (b) an exogenous nucleic acid for integration into the genome of the T cell. In some embodiments, the methods comprise (a) introducing a deletion or an insertion into the genome of a T cell using a variant Cas12i polypeptide and a first RNA guide; and (b) introducing an exogenous nucleic acid into the genome of the T cell. The Cas12i can be introduced as a ribonucleoprotein complex (e.g., Cas12i ribonucleoprotein (RNP)) with an RNA guide into a cell. The Cas12i, RNA guide, and template DNA can be introduced on a nucleic acid vector. The Cas12i can be introduced as an mRNA. The RNA guide and exogenous nucleic acid can be introduced directly into the cell.

The Cas12i polypeptide, RNA guide, and template DNA can further be delivered as described in PCT/US2021/025257. In some embodiments, the Cas12i polypeptide, RNA guide, and template DNA are delivered by transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the RNA guide is designed as described in U.S. Pat. No. 10,808,245 and PCT/US2021/025257 (WO 2021/202800), which are incorporated by reference herein in their entirety. See, e.g., the “RNA Guides” and “RNA Guide Modifications” sections of U.S. Pat. No. 10,808,245 and the “Targeting Moiety” section of PCT/US2021/025257 (WO 2021/202800).

In some embodiments wherein the Cas12i polypeptide has at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with SEQ ID NO: 3 of U.S. Pat. No. 10,808,245, the direct repeat is an RNA molecule having at least 90%, at least 95%, or 100% identity to SEQ ID NO: 7 or SEQ ID NO: 24 of U.S. Pat. No. 10,808,245 or a portion of SEQ ID NO: 7 or SEQ ID NO: 24 of U.S. Pat. No. 10,808,245. In some embodiments, the RNA guide comprises the sequence of SEQ ID NO: 156 or SEQ ID NO: 157 of U.S. Pat. No. 10,808,245 or a portion of the sequence of SEQ ID NO: 156 or SEQ ID NO: 157 of U.S. Pat. No. 10,808,245.

In some embodiments wherein the Cas12i polypeptide has at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with SEQ ID NO: 5 of U.S. Pat. No. 10,808,245 or any one of SEQ ID NOs: 2-4 or SEQ ID NOs: 46-48 of the present disclosure, the direct repeat is an RNA molecule having at least 90%, at least 95%, or 100% identity to SEQ ID NO: 9 or SEQ ID NO: 10 of U.S. Pat. No. 10,808,245 or a portion of SEQ ID NO: 9 or SEQ ID NO: 10 of U.S. Pat. No. 10,808,245. In some embodiments, the RNA guide comprises the sequence of SEQ ID NO: 162 or SEQ ID NO: 163 of U.S. Pat. No. 10,808,245 or a portion of the sequence of SEQ ID NO: 162 or SEQ ID NO: 163 of U.S. Pat. No. 10,808,245.

In some embodiments wherein the Cas12i polypeptide has at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with SEQ ID NO: 14 of U.S. Pat. No. 10,808,245, the direct repeat is an RNA molecule having at least 90%, at least 95%, or 100% identity to SEQ ID NO: 19 or SEQ ID NO: 21 of U.S. Pat. No. 10,808,245 or a portion of SEQ ID NO: 19 or SEQ ID NO: 21 of U.S. Pat. No. 10,808,245. In some embodiments, the RNA guide comprises the sequence of any one of SEQ ID NOs: 150, 151, or 153 of U.S. Pat. No. 10,808,245 or a portion of the sequence of any one of SEQ ID NOs: 150, 151, or 153 of U.S. Pat. No. 10,808,245.

In some embodiments wherein the Cas12i polypeptide has at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with SEQ ID NO: 16 of U.S. Pat. No. 10,808,245, the direct repeat of the is an RNA molecule having at least 90%, at least 95%, or 100% identity to SEQ ID NO: 7 or SEQ ID NO: 24 of U.S. Pat. No. 10,808,245 or a portion of SEQ ID NO: 6 or SEQ ID NO: 24 of U.S. Pat. No. 10,808,245. In some embodiments, the RNA guide comprises the sequence of SEQ ID NO: 152 or SEQ ID NO: 158 of U.S. Pat. No. 10,808,245 or a portion of the sequence of SEQ ID NO: 152 or SEQ ID NO: 158 of U.S. Pat. No. 10,808,245.

The RNA guide forms a complex with the Cas12i polypeptide and directs the Cas12i polypeptide to a target sequence adjacent to a 5′-NTTN-3′ sequence (e.g., PAM sequence) in a target nucleic acid. The RNA guide forms a complex with the Cas12i polypeptide and directs the Cas12i polypeptide to a target sequence adjacent to a 5′-NTTN-3′ sequence (e.g., PAM sequence) within or adjacent to a gene. In some embodiments, the 5′-NTTN-3′ sequence (e.g., PAM sequence) is a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the 5′-NTTN-3′ sequence is a 5′-CTTT-3′, 5′-CTTC-3′, 5′-GTTT-3′, 5′-GTTC-3′, 5′-TTTC-3′, 5′-GTTA-3′, or 5′-GTTG-3′ sequence.

In some embodiments, two or more (e.g., 2, 3, 4, 5, 6, 7, 8, 9, or more) RNA guides are used to introduce insertions into one or more (e.g., 1, 2, 3, 4, 5, 6, 7, 8, 9, or more) genes of a cell. For example, a first RNA guide can be designed to target a first gene, and a second RNA guide can be designed to target a second gene. In another example, a first RNA guide can be designed to target a first portion of a gene, and a second RNA guide can be designed to target a second portion of the gene.

In some embodiments, the Cas12i polypeptide has enzymatic activity (e.g., nuclease activity). In some embodiments, the Cas12i polypeptide induces one or more DNA double-stranded breaks in the cell. In some embodiments, the Cas12i polypeptide induces one or more DNA single-stranded breaks in the cell. In some embodiments, the Cas12i polypeptide induces one or more DNA nicks in the cell. In some embodiments, DNA breaks and/or nicks result in formation of one or more indels (e.g., one or more deletions or one or more insertions).

### Deletion

In some embodiments, the Cas12i polypeptide induces a deletion 3′ of a 5′-NTTN-3′ sequence on a target strand within or adjacent to a gene, relative to a reference sequence. In some embodiments, the Cas12i polypeptide induces a deletion that starts within about 5 to about 25 nucleotides of a 5′-NTTN-3′ sequence on a target strand within or adjacent to a gene, relative to a reference sequence. In some embodiments, the Cas12i polypeptide induces a deletion that starts within about 5 to about 25 nucleotides downstream or 3′ of a 5′-NTTN-3′ sequence on a target strand within or adjacent to a gene, relative to a reference sequence and ends about 15 to about 50 nucleotides downstream of the 5′-NTTN-3′ sequence. In some embodiments, the Cas12i polypeptide induces a deletion that starts within about 5 to about 25 nucleotides downstream of a 5′-NTTN-3′ sequence on a target strand within or adjacent to a gene, relative to a reference sequence and ends within about 5 to about 25 nucleotides of a 5′-NTTN-3′ sequence on the other strand, wherein the other strand 5′-NTTN-3′ sequence relative to the target strand 5′-NTTN-3′ sequence is downstream of the target strand 5′-NTTN-3′ sequence. In some embodiments, the Cas12i polypeptide induces a deletion that starts about 5 to about 25 nucleotides downstream of a 5′-NTTN-3′ sequence on a target strand within or adjacent to a gene, relative to a reference sequence and ends within about 5 to about 25 nucleotides upstream or 5′ to a complementary sequence of a 5′-NTTN-3′ sequence on the target strand, wherein the complementary 5′-NTTN-3′ sequence relative to the target strand 5′-NTTN-3′ sequence is downstream of the target strand 5′-NTTN-3′ sequence.

In some embodiments, the Cas12i polypeptide induces a deletion adjacent to (e.g., downstream of or 3′ of) a 5′-NTTN-3′ sequence, wherein N is any nucleotide. In some embodiments, the Cas12i polypeptide induces a deletion adjacent to (e.g., downstream of) a 5′-NTTN-3′ sequence within or adjacent to a gene, relative to a reference sequence, wherein N is any nucleotide. In some embodiments, the deletion is adjacent to (e.g., downstream of) a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the deletion is adjacent to (e.g., downstream of) a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the deletion is adjacent to (e.g., downstream of) a T/C-rich sequence.

In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence within or adjacent to a gene, relative to a reference sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a T/C-rich sequence.

In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence within or adjacent to a gene, relative to a reference sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a T/C-rich sequence.

In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence within or adjacent to a gene, relative to a reference sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a T/C-rich sequence.

In some embodiments, the Cas12i polypeptide induces a deletion starting within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence within or adjacent to a gene, relative to a reference sequence. In some embodiments, the Cas12i polypeptide induces a deletion starting within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the Cas12i polypeptide induces a deletion ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptide induces a deletion ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence within or adjacent to a gene, relative to a reference sequence. In some embodiments, the Cas12i polypeptide induces a deletion ending within (e.g., downstream of) about 20 to about 25 nucleotides (e.g., about 17, 18, 19, 20, 21, 22, 23, 24, 25, 26, 27, or 28 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion ending within (e.g., downstream of) about 20 to about 25 nucleotides (e.g., about 17, 18, 19, 20, 21, 22, 23, 24, 25, 26, 27, or 28 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion ending within (e.g., downstream of) about 20 to about 25 nucleotides (e.g., about 17, 18, 19, 20, 21, 22, 23, 24, 25, 26, 27, or 28 nucleotides) of a T/C-rich sequence.

In some embodiments, the Cas12i polypeptide induces a deletion ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptide induces a deletion ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence within or adjacent to a gene, relative to a reference sequence. In some embodiments, the Cas12i polypeptide induces a deletion ending within (e.g., downstream of) about 25 to about 30 nucleotides (e.g., about 22, 23, 24, 25, 26, 27, 28, 29, 30, 31, 32, or 33 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion ending within (e.g., downstream of) about 25 to about 30 nucleotides (e.g., about 22, 23, 24, 25, 26, 27, 28, 29, 30, 31, 32, or 33 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion ending within (e.g., downstream of) about 25 to about 30 nucleotides (e.g., about 22, 23, 24, 25, 26, 27, 28, 29, 30, 31, 32, or 33 nucleotides) of a T/C-rich sequence.

In some embodiments, the Cas12i polypeptide induces a deletion starting within about 5 to about 15 nucleotides (e.g., about 3, 4, 5, 6, 7, 8, 9, 10, 11, 12, 13, 14, 15, 16, or 17 nucleotides) downstream of a 5′-NTTN-3′ sequence on a target strand within or adjacent to a gene, relative to a reference sequence and ending within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the Cas12i polypeptide induces a deletion starting within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on a target strand within or adjacent to a gene, relative to a reference sequence and ending within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on the other strand or upstream of a complementary sequence to a 5′-NTTN-3′ sequence on the target strand, wherein the other strand 5′-NTTN-3′ sequence or the complementary 5′-NTTN-3′ sequence relative to the target strand 5′-NTTN-3′ sequence is downstream of the target strand 5′-NTTN-3′ sequence.

In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence within or adjacent to a gene, relative to a reference sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a T/C-rich sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the T/C-rich sequence.

In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence within or adjacent to a gene, relative to a reference sequence and ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a T/C-rich sequence and ending within (e.g., downstream of) about 20 to about 25 nucleotides (e.g., about 17, 18, 19, 20, 21, 22, 23, 24, 25, 26, 27, or 28 nucleotides) of the T/C-rich sequence.

In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence within or adjacent to a gene, relative to a reference sequence and ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a T/C-rich sequence and ending within (e.g., downstream of) about 25 to about 30 nucleotides (e.g., about 22, 23, 24, 25, 26, 27, 28, 29, 30, 31, 32, or 33 nucleotides) of the T/C-rich sequence.

In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence within or adjacent to a gene, relative to a reference sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a T/C-rich sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the T/C-rich sequence.

In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence within or adjacent to a gene, relative to a reference sequence and ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 5 to about 10 nucleotides of a T/C-rich sequence and ending within (e.g., downstream of) about 20 to about 25 nucleotides (e.g., about 17, 18, 19, 20, 21, 22, 23, 24, 25, 26, 27, or 28 nucleotides) (e.g., about 3, 4, 5, 6, 7, 8, 9, 10, 11, or 12 nucleotides) of the T/C-rich sequence.

In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence within or adjacent to a gene, relative to a reference sequence and ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a T/C-rich sequence and ending within (e.g., downstream of) about 25 to about 30 nucleotides (e.g., about 22, 23, 24, 25, 26, 27, 28, 29, 30, 31, 32, or 33 nucleotides) of the T/C-rich sequence.

In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence within or adjacent to a gene, relative to a reference sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a T/C-rich sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the T/C-rich sequence.

In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence within or adjacent to a gene, relative to a reference sequence and ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a T/C-rich sequence and ending within (e.g., downstream of) about 20 to about 25 nucleotides (e.g., about 17, 18, 19, 20, 21, 22, 23, 24, 25, 26, 27, or 28 nucleotides) of the T/C-rich sequence.

In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence within or adjacent to a gene, relative to a reference sequence and ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a 5′-CTTT-3′, 5′-CTTC-3′, 5′-GTTT-3′, 5′-GTTC-3′, 5′-TTTC-3′, 5′-GTTA-3′, or 5′-GTTG-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-CTTT-3′, 5′-CTTC-3′, 5′-GTTT-3′, 5′-GTTC-3′, 5′-TTTC-3′, 5′-GTTA-3′, or 5′-GTTG-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a T/C-rich sequence and ending within (e.g., downstream of) about 25 to about 30 nucleotides (e.g., about 22, 23, 24, 25, 26, 27, 28, 29, 30, 31, 32, or 33 nucleotides) of the T/C-rich sequence.

In some embodiments, the Cas12i polypeptide induces a deletion up to about 40 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides).

In some embodiments, the Cas12i polypeptide induces a deletion of between about 4 nucleotides and about 40 nucleotides in length (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides).

In some embodiments, the Cas12i polypeptide induces a deletion of between about 4 nucleotides and about 25 nucleotides in length (e.g., about 3, 4, 5, 6, 7, 8, 9, 10, 11, 12, 13, 14, 15, 16, 17, 18, 19, 20, 21, 22, 23, 24, 25, 26, 27, or 28 nucleotides).

In some embodiments, the Cas12i polypeptide induces a deletion of between about 10 nucleotides and about 25 nucleotides in length (e.g., about 7, 8, 9, 10, 11, 12, 13, 14, 15, 16, 17, 18, 19, 20, 21, 22, 23, 24, 25, 26, 27, or 28 nucleotides).

In some embodiments, the Cas12i polypeptide induces a deletion of between about 10 nucleotides and about 15 nucleotides in length (e.g., about 7, 8, 9, 10, 11, 12, 13, 14, 15, 16, or 17 nucleotides).

### Insertion

In some embodiments, the Cas12i polypeptide induces an insertion 3′ of a 5′-NTTN-3′ sequence on a target strand within or adjacent to a gene, relative to a reference sequence. In some embodiments, the Cas12i polypeptide induces an insertion that starts within about 5 to about 25 nucleotides of a 5′-NTTN-3′ sequence on a target strand within or adjacent to a gene, relative to a reference sequence.

In some embodiments, the Cas12i polypeptide induces an insertion adjacent to a 5′-NTTN-3′ sequence, wherein N is any nucleotide. In some embodiments, the Cas12i polypeptide induces an insertion adjacent to a 5′-NTTN-3′ sequence within or adjacent to a gene, relative to a reference sequence, wherein N is any nucleotide. In some embodiments, the Cas12i polypeptide-induced insertion is adjacent to a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide-induced insertion is adjacent to a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide-induced insertion is adjacent to a T/C-rich sequence.

In some embodiments, the Cas12i polypeptide induces an insertion downstream or 3′ of a 5′-NTTN-3′ sequence, wherein N is any nucleotide. In some embodiments, the Cas12i polypeptide induces an insertion downstream of a 5′-NTTN-3′ sequence within or adjacent to a gene, relative to a reference sequence, wherein N is any nucleotide. In some embodiments, the Cas12i polypeptide-induced insertion is downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide-induced insertion is downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide-induced insertion is downstream of a T/C-rich sequence.

In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of the 5′-NTTN-3′ sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of the 5′-NTTN-3′ sequence within or adjacent to a gene, relative to a reference sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of a T/C-rich sequence.

In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of the 5′-NTTN-3′ sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of the 5′-NTTN-3′ sequence within or adjacent to a gene, relative to a reference sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of a T/C-rich sequence.

In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) of the 5′-NTTN-3′ sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) of the 5′-NTTN-3′ sequence within or adjacent to a gene, relative to a reference sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) of a T/C-rich sequence.

In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) downstream of the 5′-NTTN-3′ sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) downstream of the 5′-NTTN-3′ sequence within or adjacent to a gene, relative to a reference sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) downstream of a T/C-rich sequence.

In some embodiments, the Cas12i polypeptide induces an insertion starting within about 20 to about 28 nucleotides (e.g., about 20, 21, 22, 23, 24, 25, 26, 27, or 28 nucleotides) of the 5′-NTTN-3′ sequence.

In some embodiments, the Cas12i polypeptide induces an insertion starting within about 20 to about 28 nucleotides (e.g., about 20, 21, 22, 23, 24, 25, 26, 27, or 28 nucleotides) of the 5′-NTTN-3′ sequence within or adjacent to a gene, relative to a reference sequence. In some embodiments, the Cas12i polypeptide induces an insertion starting within about 20 to about 28 nucleotides (e.g., about 20, 21, 22, 23, 24, 25, 26, 27, or 28 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces an insertion starting within about 20 to about 28 nucleotides (e.g., about 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide induces an insertion starting within about 20 to about 28 nucleotides (e.g., about 20, 21, 22, 23, 24, 25, 26, 27, or 28 nucleotides) of a T/C-rich sequence.

In some embodiments, the Cas12i polypeptide induces an insertion starting within about 20 to about 28 nucleotides (e.g., about 20, 21, 22, 23, 24, 25, 26, 27, or 28 nucleotides) downstream of the 5′-NTTN-3′ sequence. In some embodiments, the Cas12i polypeptide induces an insertion starting within about 20 to about 28 nucleotides (e.g., about 20, 21, 22, 23, 24, 25, 26, 27, or 28 nucleotides) downstream of the 5′-NTTN-3′ sequence within or adjacent to a gene, relative to a reference sequence. In some embodiments, the Cas12i polypeptide induces an insertion starting within about 20 to about 28 nucleotides (e.g., about 20, 21, 22, 23, 24, 25, 26, 27, or 28 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces an insertion starting within about 20 to about 28 nucleotides (e.g., about 20, 21, 22, 23, 24, 25, 26, 27, or 28 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide induces an insertion starting within about 20 to about 28 nucleotides (e.g., about 20, 21, 22, 23, 24, 25, 26, 27, or 28 nucleotides) downstream of a T/C-rich sequence.

In some embodiments, the Cas12i polypeptide induces an insertion starting within about 20 to about 25 nucleotides (e.g., about 20, 21, 22, 23, 24, or 25 nucleotides) of the 5′-NTTN-3′ sequence. In some embodiments, the Cas12i polypeptide induces an insertion starting within about 20 to about 25 nucleotides (e.g., about 20, 21, 22, 23, 24, or 25 nucleotides) of the 5′-NTTN-3′ sequence within or adjacent to a gene, relative to a reference sequence. In some embodiments, the Cas12i polypeptide induces an insertion starting within about 20 to about 25 nucleotides (e.g., about 20, 21, 22, 23, 24, or 25 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces an insertion starting within about 20 to about 25 nucleotides (e.g., about 20, 21, 22, 23, 24, or 25 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide induces an insertion starting within about 20 to about 25 nucleotides (e.g., about 20, 21, 22, 23, 24, or 25 nucleotides) of a T/C-rich sequence.

In some embodiments, the Cas12i polypeptide induces an insertion starting within about 20 to about 25 nucleotides (e.g., about 20, 21, 22, 23, 24, or 25 nucleotides) downstream of the 5′-NTTN-3′ sequence. In some embodiments, the Cas12i polypeptide induces an insertion starting within about 20 to about 25 nucleotides (e.g., about 20, 21, 22, 23, 24, or 25 nucleotides) downstream of the 5′-NTTN-3′ sequence within or adjacent to a gene, relative to a reference sequence. In some embodiments, the Cas12i polypeptide induces an insertion starting within about 20 to about 25 nucleotides (e.g., about 20, 21, 22, 23, 24, or 25 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide induces an insertion starting within about 20 to about 25 nucleotides (e.g., about 20, 21, 22, 23, 24, or 25 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide induces an insertion starting within about 20 to about 25 nucleotides (e.g., about 20, 21, 22, 23, 24, or 25 nucleotides) downstream of a T/C-rich sequence.

In some embodiments, the Cas12i polypeptide induces an insertion starting within about 5 to about 15 nucleotides (e.g., about 3, 4, 5, 6, 7, 8, 9, 10, 11, 12, 13, 14, 15, 16, or 17 nucleotides) downstream of a 5′-NTTN-3′ sequence on a target strand within or adjacent to a gene, relative to a reference sequence. In some embodiments, the Cas12i polypeptide induces an insertion starting within about 5 to about 25 nucleotides (e.g., about 3, 4, 5, 6, 7, 8, 9, 10, 11, 12, 13, 14, 15, 16, 17, 18, 19, 20, 21, 22, 23, 24, 25, 26, 27, or 28 nucleotides) downstream of a 5′-NTTN-3′ sequence on target strand within or adjacent to a gene, relative to a reference sequence. In some embodiments, the Cas12i polypeptide induces an insertion starting within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on target strand within or adjacent to a gene, relative to a reference sequence.

In some embodiments, the Cas12i polypeptide induces an insertion of up to about 9 nucleotides (e.g., about 1, 2, 3, 4, 5, 6, 7, 8, or 9 nucleotides) in length. In some embodiments, the Cas12i polypeptide induces a 1-nucleotide insertion. In some embodiments, the Cas12i polypeptide induces a 2-nucleotide insertion. In some embodiments, the Cas12i polypeptide induces a 3-nucleotide insertion. In some embodiments, the Cas12i polypeptide induces a 4-nucleotide insertion. In some embodiments, the Cas12i polypeptide induces a 5-nucleotide insertion. In some embodiments, the Cas12i polypeptide induces a 6-nucleotide insertion. In some embodiments, the Cas12i polypeptide induces a 7-nucleotide insertion. In some embodiments, the Cas12i polypeptide induces an 8-nucleotide insertion. In some embodiments, the Cas12i polypeptide induces a 9-nucleotide insertion. In some embodiments, the Cas12i polypeptide induces an insertion having a length greater than 9 nucleotides.

The disclosure also provides methods of obtaining a plurality of modified cells of the disclosure. In some embodiments, the modified cell described above is identified, isolated and cultured to produce a plurality of identical modified cells. The modified cell can be isolated using methods known in the art, e.g., by immunomagnetic cell separation, fluorescence-activated cell sorting, density gradient centrifugation, immunodensity cell separation, sedimentation, adhesion, or microfluidic cell separation. In some embodiments, a plurality of modified cells comprising the deletion and/or insertion described above is produced via introduction of the Cas12i polypeptide and RNA guide at high frequency, such that the modified cells represent at least 10%, 15%, 20%, 25%, 30%, 35%, 40%, 45%, 50%, 55%, 60%, 65%, 70%, or more of the cells present.

In some embodiments, an exogenous nucleic acid (e.g., an exogenous nucleic acid encoding a CAR) is introduced into a cell. In some embodiments, the exogenous nucleic acid is introduced into the cell through viral delivery, e.g., lentivirus or AAV. In some embodiments, the template DNA is designed as described in U.S. Pat. No. 10,808,245 and PCT/US2021/025257 (WO 2021/202800), which are incorporated by reference herein in their entirety. See, e.g., the “Methods of Using CRISPR Systems” and “Therapeutic Applications” sections and Example 10 of U.S. Pat. No. 10,808,245 and the “Targeting Moiety” section of PCT/US2021/025257 (WO 2021/202800). In some embodiments, an exogenous nucleic acid is integrated into the genome of a cell, e.g., a template DNA is integrated into the genome of a cell. In some embodiments, an exogenous nucleic acid is integrated into a gene of a cell. In some embodiments, an exogenous nucleic acid is integrated into an exon and/or intron of a gene. In some embodiments, an exogenous nucleic acid is integrated into the TRAC gene. In some embodiments, an exogenous nucleic acid is integrated into the B2M gene.

In some embodiments, the template DNA is a single-stranded nucleic acid. In some embodiments, the template DNA is a double-stranded nucleic acid. In some embodiments, the template DNA is a DNA, RNA, or DNA/RNA hybrid molecule. In some embodiments, the template DNA is a single-stranded oligo DNA nucleotide (ssODN) template DNA or comprises ssODNs. In some embodiments, the template DNA is a double-stranded oligo DNA nucleotide (dsODN) template DNA or comprises dsODNs. In some embodiments, the template DNA is linear. In some embodiments, the template DNA is circular (e.g., a plasmid). In some embodiments, the template DNA is an exogenous nucleic acid molecule, e.g., exogenous to the target cell. In some embodiments, the template DNA is a chromatid (e.g., a sister chromatid).

In some embodiments, the template DNA and the target nucleic acid are not identical in sequence. In some embodiments, a template DNA comprises one or more nucleotides that are heterologous (e.g., not homologous) to the target nucleic acid. In some embodiments, the template DNA comprises an insertion, a deletion, a polymorphism, an inversion, or a rearrangement relative to the target nucleic acid. The insertion may comprise a restriction site or a selectable marker. In some embodiments, a break in the target nucleic acid (e.g., a break induced by a Cas12i polypeptide) is repaired by HDR using the template DNA. As such, use of a template DNA can result in an insertion, deletion, or substitution in the target nucleic acid by way of HDR. In some embodiments, the insertion may comprise a gene, e.g., a wild-type gene, or a portion thereof. In some embodiments, the template DNA encodes a CAR, and a CAR is introduced into the target nucleic acid by way of HDR.

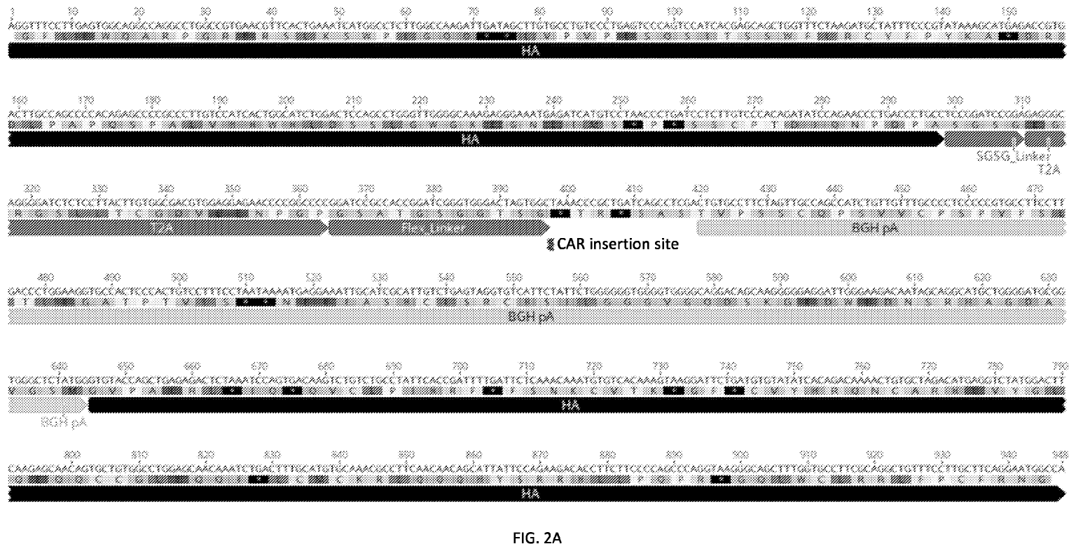

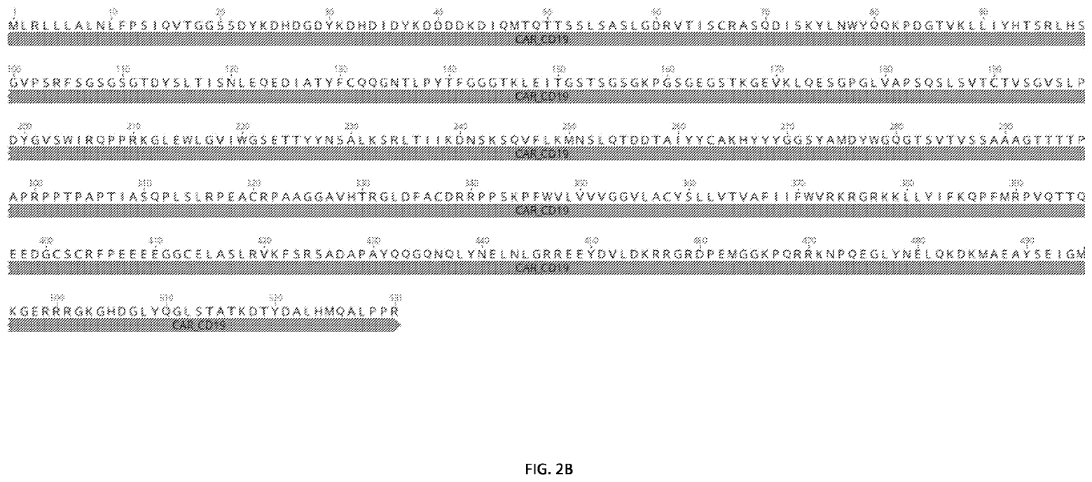

In some embodiments, the template DNA is an exogenous nucleic acid comprising one or more of a homology arm, a promoter, a linker, an open reading frame, and a polyadenylation signal, as depicted in FIG. 2A. In some embodiments, the template DNA comprises a right homology arm and a left homology arm. In certain embodiments, the left homology arm is the same length as or is about the same length as the right homology arm. In some embodiments, the left homology arm is about 10-30, 20-40, 30-50, 40-60, 50-80, 70-100, 90-150, 140-200, 190-250, 240-300, 290-350, 340-400, 390-450, or 440-500 nucleotides in length. In some embodiments, the left homology arm is about 20-200 (e.g., about 20-25, 25-30, 30-35, 35-40, 40-45, 45-50, 40-60, 50-80, 70-100, 90-150, 140-200, 190-250, 240-300, 290-350, 340-400, 390-450, or 440-500 nucleotides in length. In some embodiments, the left homology arm is about 20-200 (e.g., about 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, or 195-200 nucleotides), about 200-500 (e.g., about 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-430, 430-440, 440-450, 450-460, 460-470, 470-480, 480-490, or 490-500) nucleotides in length.

In some embodiments, the right homology arm is about 10-30, 20-40, 30-50, 40-60, 60-80, 80-100, 100-150, 150-200, 200-250, 250-300, 300-350, 350-400, 400-450, or 450-500 nucleotides in length. In certain embodiments, the right homology arm is about 20-200 (e.g., about 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, or 195-200 nucleotides), about 200-500 (e.g., about 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-430, 430-440, 440-450, 450-460, 460-470, 470-480, 480-490, or 490-500) nucleotides in length.

In some embodiments, the left homology arm comprises one or more internucleoside modifications (e.g., phosphorothioate modifications). In certain embodiments, the right homology arm comprises one or more internucleoside modifications (e.g., phosphorothioate modifications). In other embodiments, the left homology arm comprises one or more internucleoside modifications (e.g., phosphorothioate modifications) and the right homology arm comprises one or more internucleoside modifications (e.g., phosphorothioate modifications). In some embodiments, the left homology arm comprises two internucleoside modifications (e.g., phosphorothioate modifications) and the right homology arm comprises two internucleoside modifications (e.g., phosphorothioate modifications). In some embodiments, the phosphorothioate modifications are at the 5′ end of the left homology arm and the 3′ end of the right homology arm. In some embodiments, the left homology arm is 5′ of the right homology arm and the left homology arm comprises two phosphorothioate modifications at the 5′ end of the left homology arm, the right homology arm comprises two phosphorothioate modifications at the 3′ end of the right homology arm.

The disclosure also provides methods of obtaining a plurality of modified cells (e.g., modified T cells) of the disclosure. In some embodiments, the modified cell described above is identified, isolated and cultured to produce a plurality of identical modified cells.

The RNA guide forms a complex with the Cas12i polypeptide and directs the Cas12i polypeptide to a target sequence adjacent to a 5′-NTTN-3′ sequence of a target nucleic acid. The template DNA comprises an insertion (e.g., a sequence encoding a protein, e.g., a sequence encoding a CAR) relative to the target nucleic acid, and the insertion is introduced adjacent to the 5′-NTTN-3′ sequence. In some embodiments, the 5′-NTTN-3′ sequence is a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the 5′-NTTN-3′ sequence is a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence.

In some embodiments, a composition comprising a Cas12i polypeptide, an RNA guide, and a template DNA induces an insertion adjacent to a 5′-NTTN-3′ sequence, wherein N is any nucleotide. In some embodiments, the insertion is adjacent to a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the insertion is adjacent to a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the insertion is adjacent to a T/C-rich sequence.

In some embodiments, a composition comprising a Cas12i polypeptide, an RNA guide, and a template DNA induces an insertion downstream of a 5′-NTTN-3′ sequence, wherein N is any nucleotide. In some embodiments, the insertion is downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the insertion is downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the insertion is downstream of a T/C-rich sequence.

In some embodiments, a composition comprising a Cas12i polypeptide, an RNA guide, and a template DNA induces an insertion upstream of a 5′-NTTN-3′ sequence. In some embodiments, the insertion is upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the insertion is upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the insertion is upstream of a T/C-rich sequence.

In some embodiments, a composition comprising a Cas12i polypeptide, an RNA guide, and a template DNA induces an insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) of the 5′-NTTN-3′ sequence. In some embodiments, a composition comprising a Cas12i polypeptide, an RNA guide, and a template DNA induces an insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) of a T/C-rich sequence.

In some embodiments, a composition comprising a Cas12i polypeptide, an RNA guide, and a template DNA induces an insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) downstream of the 5′-NTTN-3′ sequence. In some embodiments, a composition comprising a Cas12i polypeptide, an RNA guide, and a template DNA induces an insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) downstream of a T/C-rich sequence.

In some embodiments, a composition comprising a Cas12i polypeptide, an RNA guide, and a template DNA induces an insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) upstream of the 5′-NTTN-3′ sequence. In some embodiments, a composition comprising a Cas12i polypeptide, an RNA guide, and a template DNA induces an insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5′-DTTR′3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) upstream of a T/C-rich sequence.

In some embodiments, a composition comprising a Cas12i polypeptide, an RNA guide, and a template DNA induces an insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) of the 5′-NTTN-3′ sequence. In some embodiments, a composition comprising a Cas12i polypeptide, an RNA guide, and a template DNA induces an insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) of a T/C-rich sequence.

In some embodiments, a composition comprising a Cas12i polypeptide, an RNA guide, and a template DNA induces an insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of the 5′-NTTN-3′ sequence. In some embodiments, a composition comprising a Cas12i polypeptide, an RNA guide, and a template DNA induces an insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a T/C-rich sequence.

In some embodiments, a composition comprising a Cas12i polypeptide, an RNA guide, and a template DNA induces an insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) upstream of the 5′-NTTN-3′ sequence. In some embodiments, a composition comprising a Cas12i polypeptide, an RNA guide, and a template DNA induces an insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) upstream of a T/C-rich sequence.

In some embodiments, a composition comprising a Cas12i polypeptide, an RNA guide, and a template DNA induces an insertion starting within about 3 nucleotides (e.g., 1, 2 or 3 nucleotides) upstream to about 35 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of the 5′-NTTN-3′ sequence. In some embodiments, a composition comprising a Cas12i polypeptide, an RNA guide, and a template DNA induces an insertion starting within about 3 nucleotides (e.g., 1, 2 or 3 nucleotides) upstream to about 35 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 3 nucleotides (e.g., 1, 2, or 3 nucleotides) upstream to about 35 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 3 nucleotides (e.g., 1, 2, or 3 nucleotides) upstream to about 35 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a T/C-rich sequence.

In some embodiments, a composition comprising a Cas12i polypeptide, an RNA guide, and a template DNA induces an insertion starting within about 3 to about 15 nucleotides (e.g., about 3, 4, 5, 6, 7, 8, 9, 10, 11, 12, 13, 14, or 15 nucleotides) of the 5′-NTTN-3′ sequence. In some embodiments, a composition comprising a Cas12i polypeptide, an RNA guide, and a template DNA induces an insertion starting within about 3 to about 15 nucleotides (e.g., about 3, 4, 5, 6, 7, 8, 9, 10, 11, 12, 13, 14, or 15 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 3 to about 15 nucleotides (e.g., about 3, 4, 5, 6, 7, 8, 9, 10, 11, 12, 13, 14, or 15 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 3 to about 15 nucleotides (e.g., about 3, 4, 5, 6, 7, 8, 9, 10, 11, 12, 13, 14, or 15 nucleotides) of a T/C-rich sequence.

In some embodiments, a composition comprising a Cas12i polypeptide, an RNA guide, and a template DNA induces an insertion starting within about 3 to about 15 nucleotides (e.g., about 3, 4, 5, 6, 7, 8, 9, 10, 11, 12, 13, 14, or 15 nucleotides) downstream of the 5′-NTTN-3′ sequence. In some embodiments, a composition comprising a Cas12i polypeptide, an RNA guide, and a template DNA induces an insertion starting within about 3 to about 15 nucleotides (e.g., about 3, 4, 5, 6, 7, 8, 9, 10, 11, 12, 13, 14, or 15 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 3 to about 15 nucleotides (e.g., about 3, 4, 5, 6, 7, 8, 9, 10, 11, 12, 13, 14, or 15 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 3 to about 15 nucleotides (e.g., about 3, 4, 5, 6, 7, 8, 9, 10, 11, 12, 13, 14, or 15 nucleotides) downstream of a T/C-rich sequence.

In some embodiments, a composition comprising a Cas12i polypeptide, an RNA guide, and a template DNA induces an insertion starting within about 3 to about 15 nucleotides (e.g., about 3, 4, 5, 6, 7, 8, 9, 10, 11, 12, 13, 14, or 15 nucleotides) upstream of the 5′-NTTN-3′ sequence. In some embodiments, a composition comprising a Cas12i polypeptide, an RNA guide, and a template DNA induces an insertion starting within about 3 to about 15 nucleotides (e.g., about 3, 4, 5, 6, 7, 8, 9, 10, 11, 12, 13, 14, or 15 nucleotides) upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 3 to about 15 nucleotides (e.g., about 3, 4, 5, 6, 7, 8, 9, 10, 11, 12, 13, 14, or 15 nucleotides) upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 3 to about 15 nucleotides (e.g., about 3, 4, 5, 6, 7, 8, 9, 10, 11, 12, 13, 14, or 15 nucleotides) upstream of a T/C-rich sequence.

In some embodiments, a composition comprising a Cas12i polypeptide, an RNA guide, and a template DNA induces an insertion starting within about 15 to about 30 nucleotides (e.g., about 15, 16, 17, 18, 19, 20, 21, 22, 23, 24, 25, 26, 27, 28, 29, or 30 nucleotides) of the 5′-NTTN-3′ sequence. In some embodiments, a composition comprising a Cas12i polypeptide, an RNA guide, and a template DNA induces an insertion starting within about 15 to about 30 nucleotides (e.g., about 15, 16, 17, 18, 19, 20, 21, 22, 23, 24, 25, 26, 27, 28, 29, or 30 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 15 to about 30 nucleotides (e.g., about 15, 16, 17, 18, 19, 20, 21, 22, 23, 24, 25, 26, 27, 28, 29, or 30 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 15 to about 30 nucleotides (e.g., about 15, 16, 17, 18, 19, 20, 21, 22, 23, 24, 25, 26, 27, 28, 29, or 30 nucleotides) of a T/C-rich sequence.

In some embodiments, a composition comprising a Cas12i polypeptide, an RNA guide, and a template DNA induces an insertion starting within about 15 to about 30 nucleotides (e.g., about 15, 16, 17, 18, 19, 20, 21, 22, 23, 24, 25, 26, 27, 28, 29, or 30 nucleotides) downstream of the 5′-NTTN-3′ sequence. In some embodiments, a composition comprising a Cas12i polypeptide, an RNA guide, and a template DNA induces an insertion starting within about 15 to about 30 nucleotides (e.g., about 15, 16, 17, 18, 19, 20, 21, 22, 23, 24, 25, 26, 27, 28, 29, or 30 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 15 to about 30 nucleotides (e.g., about 15, 16, 17, 18, 19, 20, 21, 22, 23, 24, 25, 26, 27, 28, 29, or 30 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 15 to about 30 nucleotides (e.g., about 15, 16, 17, 18, 19, 20, 21, 22, 23, 24, 25, 26, 27, 28, 29, or 30 nucleotides) downstream of a T/C-rich sequence.

In some embodiments, a composition comprising a Cas12i polypeptide, an RNA guide, and a template DNA induces an insertion starting within about 15 to about 30 nucleotides (e.g., about 15, 16, 17, 18, 19, 20, 21, 22, 23, 24, 25, 26, 27, 28, 29, or 30 nucleotides) upstream of the 5′-NTTN-3′ sequence. In some embodiments, a composition comprising a Cas12i polypeptide, an RNA guide, and a template DNA induces an insertion starting within about 15 to about 30 nucleotides (e.g., about 15, 16, 17, 18, 19, 20, 21, 22, 23, 24, 25, 26, 27, 28, 29, or 30 nucleotides) upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces an insertion starting within about 15 to about 30 nucleotides (e.g., about 15, 16, 17, 18, 19, 20, 21, 22, 23, 24, 25, 26, 27, 28, 29, or 30 nucleotides) upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces an insertion starting within about 15 to about 30 nucleotides (e.g., about 15, 16, 17, 18, 19, 20, 21, 22, 23, 24, 25, 26, 27, 28, 29, or 30 nucleotides) upstream of a T/C-rich sequence.

In some embodiments, a composition comprising a Cas12i polypeptide, an RNA guide, and a template DNA induces an insertion of at least about 10 or more nucleotides (e.g., at least about 25, 50, 75, 100, 150, 200, 250, 300, 350, 400, 450, 500, 600, 750, 1000, 1500, 2000, 2500, or 3000 nucleotides) in length. In some embodiments, a composition comprising a Cas12i polypeptide, an RNA guide, and a template DNA induces an insertion of at least 10 nucleotides. In some embodiments, a composition comprising a Cas12i polypeptide, an RNA guide, and a template DNA induces an insertion of at least 50 nucleotides. In some embodiments, a composition comprising a Cas12i polypeptide, an RNA guide, and a template DNA induces an insertion of at least 100 nucleotides. In some embodiments, a composition comprising a Cas12i polypeptide, an RNA guide, and a template DNA induces an insertion of at least 500 nucleotides. In some embodiments, a composition comprising a Cas12i polypeptide, an RNA guide, and a template DNA induces an insertion of at least 750 nucleotides. In some embodiments, a composition comprising a Cas12i polypeptide, an RNA guide, and a template DNA induces an insertion of at least 1000 nucleotides. In some embodiments, a composition comprising a Cas12i polypeptide, an RNA guide, and a template DNA induces an insertion of at least 1500 nucleotides. In some embodiments, a composition comprising a Cas12i polypeptide, an RNA guide, and a template DNA induces an insertion of at least 2000 nucleotides. In some embodiments, a composition comprising a Cas12i polypeptide, an RNA guide, and a template DNA induces an insertion of at least 2500 nucleotides. In some embodiments, a composition comprising a Cas12i polypeptide, an RNA guide, and a template DNA induces an insertion having a length greater than 3000 nucleotides.

In some embodiments, a composition comprising a Cas12i polypeptide, an RNA guide, and a template DNA induces an insertion adjacent to a 5′-NTTN-3′ sequence in a T cell, wherein N is any nucleotide, and the insertion encodes a CAR (hereinafter a “CAR insertion”), which also optionally includes a regulatory sequence directing expression of the CAR. In some embodiments, the Cas12i polypeptide-induced CAR insertion is adjacent to a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide-induced CAR insertion is adjacent to a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide-induced CAR insertion is adjacent to a T/C-rich sequence.

In some embodiments, a composition comprising a Cas12i polypeptide, an RNA guide, and a template DNA induces a CAR insertion downstream of a 5′-NTTN-3′ sequence, wherein N is any nucleotide. In some embodiments, the Cas12i polypeptide-induced CAR insertion is downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide-induced CAR insertion is downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide-induced CAR insertion is downstream of a T/C-rich sequence.

In some embodiments, a composition comprising a Cas12i polypeptide, an RNA guide, and a template DNA induces a CAR insertion upstream of a 5′-NTTN-3′ sequence. In some embodiments, the Cas12i polypeptide-induced CAR insertion is upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, the Cas12i polypeptide-induced CAR insertion is upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the Cas12i polypeptide-induced CAR insertion is upstream of a T/C-rich sequence.

In some embodiments, a composition comprising a Cas12i polypeptide, an RNA guide, and a template DNA induces a CAR insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) of the 5′-NTTN-3′ sequence. In some embodiments, a composition comprising a Cas12i polypeptide, an RNA guide, and a template DNA induces a CAR insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) of a T/C-rich sequence.

In some embodiments, a composition comprising a Cas12i polypeptide, an RNA guide, and a template DNA induces a CAR insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) downstream of the 5′-NTTN-3′ sequence. In some embodiments, a composition comprising a Cas12i polypeptide, an RNA guide, and a template DNA induces a CAR insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) downstream of a T/C-rich sequence.

In some embodiments, a composition comprising a Cas12i polypeptide, an RNA guide, and a template DNA induces a CAR insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) upstream of the 5′-NTTN-3′ sequence. In some embodiments, a composition comprising a Cas12i polypeptide, an RNA guide, and a template DNA induces a CAR insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 3 to about 200 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 110, 120, 130, 140, 150, 160, 170, 180, 190, or 200 nucleotides) upstream of a T/C-rich sequence.

In some embodiments, a composition comprising a Cas12i polypeptide, an RNA guide, and a template DNA induces a CAR insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) of the 5′-NTTN-3′ sequence. In some embodiments, a composition comprising a Cas12i polypeptide, an RNA guide, and a template DNA induces a CAR insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) of a T/C-rich sequence.

In some embodiments, a composition comprising a Cas12i polypeptide, an RNA guide, and a template DNA induces a CAR insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of the 5′-NTTN-3′ sequence. In some embodiments, a composition comprising a Cas12i polypeptide, an RNA guide, and a template DNA induces a CAR insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a T/C-rich sequence.

In some embodiments, a composition comprising a Cas12i polypeptide, an RNA guide, and a template DNA induces a CAR insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) upstream of the 5′-NTTN-3′ sequence. In some embodiments, a composition comprising a Cas12i polypeptide, an RNA guide, and a template DNA induces a CAR insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 3 to about 35 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) upstream of a T/C-rich sequence.

In some embodiments, a composition comprising a Cas12i polypeptide, an RNA guide, and a template DNA induces a CAR insertion starting within about 3 nucleotides (e.g., 1, 2 or 3 nucleotides) upstream to about 35 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of the 5′-NTTN-3′ sequence. In some embodiments, a composition comprising a Cas12i polypeptide, an RNA guide, and a template DNA induces a CAR insertion starting within about 3 nucleotides (e.g., 1, 2 or 3 nucleotides) upstream to about 35 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 3 nucleotides (e.g., 1, 2, or 3 nucleotides) upstream to about 35 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 3 nucleotides (e.g., 1, 2, or 3 nucleotides) upstream to about 35 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides) downstream of a T/C-rich sequence.

In some embodiments, a composition comprising a Cas12i polypeptide, an RNA guide, and a template DNA induces a CAR insertion starting within about 3 to about 15 nucleotides (e.g., about 3, 4, 5, 6, 7, 8, 9, 10, 11, 12, 13, 14, or 15 nucleotides) of the 5′-NTTN-3′ sequence. In some embodiments, a composition comprising a Cas12i polypeptide, an RNA guide, and a template DNA induces a CAR insertion starting within about 3 to about 15 nucleotides (e.g., about 3, 4, 5, 6, 7, 8, 9, 10, 11, 12, 13, 14, or 15 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 3 to about 15 nucleotides (e.g., about 3, 4, 5, 6, 7, 8, 9, 10, 11, 12, 13, 14, or 15 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 3 to about 15 nucleotides (e.g., about 3, 4, 5, 6, 7, 8, 9, 10, 11, 12, 13, 14, or 15 nucleotides) of a T/C-rich sequence.

In some embodiments, a composition comprising a Cas12i polypeptide, an RNA guide, and a template DNA induces a CAR insertion starting within about 3 to about 15 nucleotides (e.g., about 3, 4, 5, 6, 7, 8, 9, 10, 11, 12, 13, 14, or 15 nucleotides) downstream of the 5′-NTTN-3′ sequence. In some embodiments, a composition comprising a Cas12i polypeptide, an RNA guide, and a template DNA induces a CAR insertion starting within about 3 to about 15 nucleotides (e.g., about 3, 4, 5, 6, 7, 8, 9, 10, 11, 12, 13, 14, or 15 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 3 to about 15 nucleotides (e.g., about 3, 4, 5, 6, 7, 8, 9, 10, 11, 12, 13, 14, or 15 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 3 to about 15 nucleotides (e.g., about 3, 4, 5, 6, 7, 8, 9, 10, 11, 12, 13, 14, or 15 nucleotides) downstream of a T/C-rich sequence.

In some embodiments, a composition comprising a Cas12i polypeptide, an RNA guide, and a template DNA induces a CAR insertion starting within about 3 to about 15 nucleotides (e.g., about 3, 4, 5, 6, 7, 8, 9, 10, 11, 12, 13, 14, or 15 nucleotides) upstream of the 5′-NTTN-3′ sequence. In some embodiments, a composition comprising a Cas12i polypeptide, an RNA guide, and a template DNA induces a CAR insertion starting within about 3 to about 15 nucleotides (e.g., about 3, 4, 5, 6, 7, 8, 9, 10, 11, 12, 13, 14, or 15 nucleotides) upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 3 to about 15 nucleotides (e.g., about 3, 4, 5, 6, 7, 8, 9, 10, 11, 12, 13, 14, or 15 nucleotides) upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 3 to about 15 nucleotides (e.g., about 3, 4, 5, 6, 7, 8, 9, 10, 11, 12, 13, 14, or 15 nucleotides) upstream of a T/C-rich sequence.

In some embodiments, a composition comprising a Cas12i polypeptide, an RNA guide, and a template DNA induces a CAR insertion starting within about 15 to about 30 nucleotides (e.g., about 15, 16, 17, 18, 19, 20, 21, 22, 23, 24, 25, 26, 27, 28, 29, or 30 nucleotides) of the 5′-NTTN-3′ sequence. In some embodiments, a composition comprising a Cas12i polypeptide, an RNA guide, and a template DNA induces a CAR insertion starting within about 15 to about 30 nucleotides (e.g., about 15, 16, 17, 18, 19, 20, 21, 22, 23, 24, 25, 26, 27, 28, 29, or 30 nucleotides) of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 15 to about 30 nucleotides (e.g., about 15, 16, 17, 18, 19, 20, 21, 22, 23, 24, 25, 26, 27, 28, 29, or 30 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 15 to about 30 nucleotides (e.g., about 15, 16, 17, 18, 19, 20, 21, 22, 23, 24, 25, 26, 27, 28, 29, or 30 nucleotides) of a T/C-rich sequence.

In some embodiments, a composition comprising a Cas12i polypeptide, an RNA guide, and a template DNA induces a CAR insertion starting within about 15 to about 30 nucleotides (e.g., about 15, 16, 17, 18, 19, 20, 21, 22, 23, 24, 25, 26, 27, 28, 29, or 30 nucleotides) downstream of the 5′-NTTN-3′ sequence. In some embodiments, a composition comprising a Cas12i polypeptide, an RNA guide, and a template DNA induces a CAR insertion starting within about 15 to about 30 nucleotides (e.g., about 15, 16, 17, 18, 19, 20, 21, 22, 23, 24, 25, 26, 27, 28, 29, or 30 nucleotides) downstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 15 to about 30 nucleotides (e.g., about 15, 16, 17, 18, 19, 20, 21, 22, 23, 24, 25, 26, 27, 28, 29, or 30 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 15 to about 30 nucleotides (e.g., about 15, 16, 17, 18, 19, 20, 21, 22, 23, 24, 25, 26, 27, 28, 29, or 30 nucleotides) downstream of a T/C-rich sequence.

In some embodiments, a composition comprising a Cas12i polypeptide, an RNA guide, and a template DNA induces a CAR insertion starting within about 15 to about 30 nucleotides (e.g., about 15, 16, 17, 18, 19, 20, 21, 22, 23, 24, 25, 26, 27, 28, 29, or 30 nucleotides) upstream of the 5′-NTTN-3′ sequence. In some embodiments, a composition comprising a Cas12i polypeptide, an RNA guide, and a template DNA induces a CAR insertion starting within about 15 to about 30 nucleotides (e.g., about 15, 16, 17, 18, 19, 20, 21, 22, 23, 24, 25, 26, 27, 28, 29, or 30 nucleotides) upstream of a 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′ sequence. In some embodiments, a composition comprising a Cas12i polypeptide, an RNA guide, and a template DNA induces a CAR insertion starting within about 15 to about 30 nucleotides (e.g., about 15, 16, 17, 18, 19, 20, 21, 22, 23, 24, 25, 26, 27, 28, 29, or 30 nucleotides) upstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, a composition comprising a Cas12i polypeptide, an RNA guide, and a template DNA induces a CAR insertion starting within about 15 to about 30 nucleotides (e.g., about 15, 16, 17, 18, 19, 20, 21, 22, 23, 24, 25, 26, 27, 28, 29, or 30 nucleotides) upstream of a T/C-rich sequence.

In some embodiments, a composition comprising a Cas12i polypeptide, an RNA guide, and a template DNA induces an insertion, e.g., a CAR insertion of at least about 1000 nucleotides (e.g., at least about 1000, 1500, 2000, 2500, or 3000 nucleotides) in length. In some embodiments, a composition comprising a Cas12i polypeptide, an RNA guide, and a template DNA induces a CAR insertion of at least 10 nucleotides. In some embodiments, a composition comprising a Cas12i polypeptide, an RNA guide, and a template DNA induces a CAR insertion of at least 50 nucleotides. In some embodiments, a composition comprising a Cas12i polypeptide, an RNA guide, and a template DNA induces a CAR insertion of at least 100 nucleotides. In some embodiments, a composition comprising a Cas12i polypeptide, an RNA guide, and a template DNA induces a CAR insertion of at least 500 nucleotides. In some embodiments, a composition comprising a Cas12i polypeptide, an RNA guide, and a template DNA induces a CAR insertion of at least 750 nucleotides. In some embodiments, a composition comprising a Cas12i polypeptide, an RNA guide, and a template DNA induces a CAR insertion of at least 1000 nucleotides. In some embodiments, a composition comprising a Cas12i polypeptide, an RNA guide, and a template DNA induces a CAR insertion of at least 1500 nucleotides. In some embodiments, a composition comprising a Cas12i polypeptide, an RNA guide, and a template DNA induces a CAR insertion of at least 2000 nucleotides. In some embodiments, a composition comprising a Cas12i polypeptide, an RNA guide, and a template DNA induces a CAR insertion of at least 2500 nucleotides. In some embodiments, a composition comprising a Cas12i polypeptide, an RNA guide, and a template DNA induces a CAR insertion having a length greater than 3000 nucleotides.

In a first aspect, the invention provides a method of making a modified T cell, the method comprising introducing into a T cell: (a) a variant Cas12i polypeptide or a nucleic acid encoding the variant Cas12i polypeptide, (b) a first RNA guide or a nucleic acid encoding the first RNA guide, and (c) an exogenous nucleic acid for integration into the genome of the T cell.

In some embodiments, the variant Cas12i polypeptide is a variant Cas12i2 polypeptide. In some embodiments, the variant Cas12i polypeptide comprises a sequence having at least 90% identity to a sequence of any one of SEQ ID NOs: 3-7. In some embodiments, the variant Cas12i polypeptide comprises a sequence of any one of SEQ ID NOs: 3-7.

In some embodiments, the first RNA guide comprises a spacer sequence specific to a TRAC gene or to a B2M gene. In some embodiments, the TRAC gene or the B2M gene is disrupted. In some embodiments, the disruption is a deletion. In some embodiments, the disruption is an insertion. In some embodiments, the disruption occurs in one or both alleles of the TRAC gene or the B2M gene. In some embodiments, the disruption inhibits or decreases expression of the TRAC gene or the B2M gene. In some embodiments, the disruption is within 100 nucleotides of a 5′-NTTN-3′ sequence.

In some embodiments, the exogenous nucleic acid is integrated into the genome. In some embodiments, the exogenous nucleic acid is integrated into the TRAC gene or the B2M gene. In some embodiments, the exogenous nucleic acid comprises a sequence encoding a protein. In some embodiments, the exogenous nucleic acid comprises a regulatory sequence operably linked to the sequence encoding the protein. In some embodiments, the protein is a chimeric antigen receptor (CAR). In some embodiments, the CAR comprises a single chain antibody (scFv), a transmembrane domain, an intracellular signaling domain, and, optionally, a hinge domain between the scFv and the transmembrane domain. In some embodiments, the intracellular signaling domain comprises a CD3ζ signaling domain. In some embodiments, the intracellular signaling domain further comprises a signaling domain selected from the group consisting of CD28, 4-1BB, ICOS, OX40, CD27, p56-lck, or a combination of two or more thereof. In some embodiments, the CAR comprises a scFv that is specific for CD19, CD22, BCMA, HER2, IL13Ra2, CD123, FAP, VEGFR-2, ganglioside GD2, EGFRvIII, mesothelin, or EphA. In some embodiments, the exogenous nucleic acid is integrated into the genome of the T cell by homology directed repair (HDR). In some embodiments, the exogenous nucleic acid is included in a vector. In some embodiments, the vector is a viral vector. In some embodiments, the viral vector is a lentiviral vector or an AAV vector.

In some embodiments, the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the TRAC gene. In some embodiments, the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the B2M gene.

In a related aspect, the invention provides a modified T cell generated by a method described above. In one some embodiments, the T cell is a human T cell or is derived from a human cell or a human T cell.

In a related aspect, the invention provides a composition or formulation comprising one or more modified T cell(s) of described above.

In a second aspect, the invention further provides a method of making a modified T cell, the method comprising: (a) disrupting a gene in the genome of a T cell using a variant Cas12i polypeptide and a first RNA guide, and (b) introducing an exogenous nucleic acid into the genome of the T cell.

In some embodiments, the variant Cas12i polypeptide is a variant Cas12i2 polypeptide. In some embodiments, the variant Cas12i polypeptide comprises a sequence having at least 90% identity to a sequence of any one of SEQ ID NOs: 3-7. In some embodiments, the variant Cas12i polypeptide comprises a sequence of any one of SEQ ID NOs: 3-7.

In some embodiments, the first RNA guide comprises a spacer sequence specific to a TRAC gene or to a B2M gene. In some embodiments, the TRAC gene or the B2M gene is disrupted. In some embodiments, the disruption is a deletion. In some embodiments, the disruption is an insertion. In some embodiments, the disruption occurs in one or both alleles of the TRAC gene or the B2M gene. In some embodiments, the disruption inhibits or decreases expression of the TRAC gene or the B2M gene. In some embodiments, the disruption is within 100 nucleotides of a 5′-NTTN-3′ sequence.

In some embodiments, the exogenous nucleic acid is integrated into the genome. In some embodiments, the exogenous nucleic acid is integrated into the TRAC gene or the B2M gene. In some embodiments, the exogenous nucleic acid comprises a sequence encoding a protein. In some embodiments, the exogenous nucleic acid comprises a regulatory sequence operably linked to the sequence encoding the protein. In some embodiments, the protein is a chimeric antigen receptor (CAR). In some embodiments, the CAR comprises a single chain antibody (scFv), a transmembrane domain, an intracellular signaling domain, and, optionally, a hinge domain between the scFv and the transmembrane domain. In some embodiments, the intracellular signaling domain comprises a CD3ζ signaling domain. In some embodiments, the intracellular signaling domain further comprises a signaling domain selected from the group consisting of CD28, 4-1BB, ICOS, OX40, CD27, p56-lck, or a combination of two or more thereof.

In some embodiments, the CAR comprises a scFv that is specific for CD19, CD22, BCMA, HER2, IL13Ra2, CD123, FAP, VEGFR-2, ganglioside GD2, EGFRvIII, mesothelin, or EphA. In some embodiments, the exogenous nucleic acid is integrated into the genome of the T cell by homology directed repair (HDR). In some embodiments, the exogenous nucleic acid is included in a vector. In some embodiments, the vector is a viral vector. In some embodiments, the viral vector is a lentiviral vector or an AAV vector.

In some embodiments, the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the TRAC gene. In some embodiments, the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the B2M gene.

In a related aspect, the invention provides a modified T cell generated by a method described above. In one some embodiments, the T cell is a human T cell or is derived from a human cell or a human T cell.

In a related aspect, the invention provides a composition or formulation comprising one or more modified T cell(s) of described above.

In a third aspect, the invention provides a method of making a modified T cell, the method comprising introducing into a T cell: (a) a variant Cas12i polypeptide, or a nucleic acid encoding the variant Cas12i polypeptide, and a first RNA guide, or a nucleic acid encoding the first RNA guide, for inducing a deletion or an insertion in the genome of the T cell; and (b) an exogenous nucleic acid for integration into the genome of the T cell.

In some embodiments, the variant Cas12i polypeptide is a variant Cas12i2 polypeptide. In some embodiments, the variant Cas12i polypeptide comprises a sequence having at least 90% identity to a sequence of any one of SEQ ID NOs: 3-7. In some embodiments, the variant Cas12i polypeptide comprises a sequence of any one of SEQ ID NOs: 3-7.

In some embodiments, the integration of the exogenous nucleic acid is within 100 nucleotides of a 5′-NTTN-3′ sequence. In some embodiments, the first RNA guide comprises a spacer sequence specific to a TRAC gene or to a B2M gene. In some embodiments, the deletion or the insertion is introduced into the TRAC gene or the B2M gene. In some embodiments, the deletion or the insertion is in one or both alleles of the TRAC gene or the B2M gene. In some embodiments, the deletion or the insertion inhibits or decreases expression of the TRAC gene or the B2M gene. In some embodiments, the deletion or the insertion is within 100 nucleotides of a 5′-NTTN-3′ sequence. In some embodiments, the exogenous nucleic acid is integrated into the genome. In some embodiments, the exogenous nucleic acid is integrated into the TRAC gene or the B2M gene.

In some embodiments, the exogenous nucleic acid comprises a sequence encoding a protein. In some embodiments, the exogenous nucleic acid comprises a regulatory sequence operably linked to the sequence encoding the protein. In some embodiments, the protein is a chimeric antigen receptor (CAR). In some embodiments, the CAR comprises a single chain antibody (scFv), a transmembrane domain, an intracellular signaling domain, and, optionally, a hinge domain between the scFv and the transmembrane domain. In some embodiments, the intracellular signaling domain comprises a CD3ζ signaling domain. In some embodiments, the intracellular signaling domain further comprises a signaling domain selected from the group consisting of CD28, 4-1BB, ICOS, OX40, CD27, p56-lck, or a combination of two or more thereof. In some embodiments, the CAR comprises a scFv that is specific for CD19, CD22, BCMA, HER2, IL13Ra2, CD123, FAP, VEGFR-2, ganglioside GD2, EGFRvIII, mesothelin, or EphA.

In some embodiments, the exogenous nucleic acid is integrated into the genome of the T cell by homology directed repair (HDR). In some embodiments, the exogenous nucleic acid is included in a vector. In some embodiments, the vector is a viral vector. In some embodiments, the viral vector is a lentiviral vector or an AAV vector.

In some embodiments, the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the TRAC gene. In some embodiments, the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the B2M gene.

In a related aspect, the invention provides a modified T cell generated by a method described above. In some embodiments, the T cell is a human T cell or is derived from a human cell or a human T cell.

In a related aspect, the invention provides a composition or formulation comprising one or more modified T cell(s) described above.

In a fourth aspect, the invention provides a method of making a modified T cell, the method comprising: (a) introducing a deletion or an insertion into the genome of a T cell using a variant Cas12i polypeptide and a first RNA guide; and (b) introducing an exogenous nucleic acid into the genome of the T cell.

In some embodiments, the variant Cas12i polypeptide is a variant Cas12i2 polypeptide. In some embodiments, the variant Cas12i polypeptide comprises a sequence having at least 90% identity to a sequence of any one of SEQ ID NOs: 3-7. In some embodiments, the variant Cas12i polypeptide comprises a sequence of any one of SEQ ID NOs: 3-7.

In some embodiments, the integration of the exogenous nucleic acid is within 100 nucleotides of a 5′-NTTN-3′ sequence. In some embodiments, the first RNA guide comprises a spacer sequence specific to a TRAC gene or to a B2M gene. In some embodiments, the deletion or the insertion is introduced into the TRAC gene or the B2M gene. In some embodiments, the deletion or the insertion is in one or both alleles of the TRAC gene or the B2M gene. In some embodiments, the deletion or the insertion inhibits or decreases expression of the TRAC gene or the B2M gene. In some embodiments, the deletion or the insertion is within 100 nucleotides of a 5′-NTTN-3′ sequence. In some embodiments, the exogenous nucleic acid is integrated into the genome. In some embodiments, the exogenous nucleic acid is integrated into the TRAC gene or the B2M gene.

In some embodiments, the exogenous nucleic acid comprises a sequence encoding a protein. In some embodiments, the exogenous nucleic acid comprises a regulatory sequence operably linked to the sequence encoding the protein. In some embodiments, the protein is a chimeric antigen receptor (CAR).

In some embodiments, the CAR comprises a single chain antibody (scFv), a transmembrane domain, an intracellular signaling domain, and, optionally, a hinge domain between the scFv and the transmembrane domain. In some embodiments, the intracellular signaling domain comprises a CD3ζ signaling domain. In some embodiments, the intracellular signaling domain further comprises a signaling domain selected from the group consisting of CD28, 4-1BB, ICOS, OX40, CD27, p56-lck, or a combination of two or more thereof. In some embodiments, the CAR comprises a scFv that is specific for CD19, CD22, BCMA, HER2, IL13Ra2, CD123, FAP, VEGFR-2, ganglioside GD2, EGFRvIII, mesothelin, or EphA.

In some embodiments, the exogenous nucleic acid is integrated into the genome of the T cell by homology directed repair (HDR). In some embodiments, the exogenous nucleic acid is included in a vector. In some embodiments, the vector is a viral vector. In some embodiments, the viral vector is a lentiviral vector or an AAV vector.

In some embodiments, the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the TRAC gene. In some embodiments, the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the B2M gene.

In a related aspect, the invention provides a modified T cell generated by a method described above. In some embodiments, the T cell is a human T cell or is derived from a human cell or a human T cell.

In a related aspect, the invention provides a composition or formulation comprising one or more modified T cell(s) described above.

### Compositions and Formulations

The disclosure also provides a composition or formulation comprising the modified cell (e.g., modified T cell) or plurality of modified cells (e.g., modified T cells) described herein. In some embodiments, the composition or formulation includes a cell or plurality of cells modified by Cas12i. In some embodiments, the composition or formulation includes a cell or plurality of cells comprising a deletion as described herein. In some embodiments, the composition or formulation includes a cell or plurality of cells comprising an insertion as described herein. In some embodiments, the composition or formulation includes a cell or plurality of cells comprising modifications as described herein. In some embodiments, the composition or formulation includes a cell or plurality of cells comprising an insertion, a deletion and/or a substitution as described herein. In some embodiments, the composition or formulation includes a cell or plurality of cells comprising an insertion of one or more nucleic acids as described herein. In some embodiments, the composition or formulation includes a cell line modified by Cas12i. In some embodiments, the composition or formulation includes a cell line comprising a deletion as described herein. In some embodiments, the composition or formulation includes a cell line comprising an insertion as described herein. In some embodiments, the composition or formulation includes a cell line comprising an insertion of an exogenous nucleic acid as described herein. The composition or formulation can additionally include, optionally, media and/or instructions for use of the modified cell or cell line.

In some embodiments, the composition or formulation comprises a plurality of cells (e.g., T cells) that include at least 10% modified cells (e.g., modified T cells) described herein, e.g., at least 10%, 15%, 20%, 25%, 30%, 35%, 40%, 45%, 50%, 55%, 60%, 65%, 70%, or more of the plurality are the modified cells. In some embodiments, the composition or formulation comprises a plurality of cells that include at least 70% modified cells described herein, e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more of the plurality are the modified cells. In some embodiments, the composition or formulation comprises a plurality of cells that include at least 80% modified cells described herein, e.g., at least about 80%, 81%, 82%, 83%, 84%, 85%, 86%, 87%, 88%, 89%, 90%, 91%, 92%, 93%, 94%, 95%, 96%, 97%, 98%, 99%, or more of the plurality are the modified cells. In some embodiments, the composition or formulation comprises a plurality of cells that include at least 90% modified cells described herein, e.g., at least about 90%, 91%, 92%, 93%, 94%, 95%, 96%, 97%, 98%, 99%, or more of the plurality are the modified cells.

In some embodiments, the composition is a pharmaceutical composition. A pharmaceutical composition that is useful may be prepared, packaged, or sold in a formulation suitable for oral, rectal, vaginal, parenteral, topical, pulmonary, intranasal, intra-lesional, buccal, ophthalmic, intravenous, intra-organ or another route of administration. A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined number of cells. The number of cells is generally equal to the dosage of the cells which would be administered to a subject or a convenient fraction of such a dosage such as, for example, one-half or one-third of such a dosage.

A formulation of a pharmaceutical composition suitable for parenteral administration may comprise the cells combined with a pharmaceutically acceptable carrier, such as sterile water or sterile isotonic saline. Such a formulation may be prepared, packaged, or sold in a form suitable for bolus administration or for continuous administration. Some injectable formulations may be prepared, packaged, or sold in unit dosage form, such as in ampules or in multi-dose containers containing a preservative. Some formulations for parenteral administration include, but are not limited to, suspensions, solutions, emulsions in oily or aqueous vehicles, pastes, and implantable sustained-release or biodegradable formulations. Some formulations may further comprise one or more additional ingredients including, but not limited to, suspending, stabilizing, or dispersing agents.

The pharmaceutical composition may be prepared, packaged, or sold in the form of a sterile injectable aqueous or oily suspension or solution. This suspension or solution may be formulated according to the known art, and may comprise, in addition to the cells, additional ingredients such as the dispersing agents, wetting agents, or suspending agents described herein. Such sterile injectable formulation may be prepared using a non-toxic parenterally-acceptable diluent or solvent, such as water or saline. Other acceptable diluents and solvents include, but are not limited to, Ringer's solution, isotonic sodium chloride solution, and fixed oils such as synthetic mono- or di-glycerides. Other parentally-administrable formulations which that are useful include those which may comprise the cells in a packaged form, in a liposomal preparation, or as a component of a biodegradable polymer system. Some compositions for sustained release or implantation may comprise pharmaceutically acceptable polymeric or hydrophobic materials such as an emulsion, an ion exchange resin, a sparingly soluble polymer, or a sparingly soluble salt.

### Uses

In some embodiments, the composition or formulation comprising the modified cell (e.g., modified T cell) or a plurality of the modified cells (e.g., modified T cells) as described herein may be useful for research purposes. In some embodiments, the composition or formulation comprising the modified cell or a plurality of the modified cells as described herein may be useful to study gene function. In some embodiments, the composition or formulation comprising the modified cell or a plurality of the modified cells as described herein may be useful as an expression system to manufacture biomolecules. For example, in some embodiments, the composition or formulation comprising the modified cell or a plurality of the modified cells as described herein may be useful to produce biomolecules such as proteins (e.g., cytokines, antibodies, antibody-based molecules), peptides, lipids, carbohydrates, nucleic acids, amino acids, and vitamins. In other embodiments, the composition or formulation comprising the modified cell or a plurality of the modified cells as described herein may be useful in the production of a viral vector such as a lentivirus, adenovirus, adeno-associated virus, and oncolytic virus vector. In some embodiments, the composition or formulation comprising the modified cell or a plurality of the modified cells as described herein may be useful in cytotoxicity studies. In some embodiments, the composition or formulation comprising the modified cell or a plurality of the modified cells as described herein may be useful as a disease model. In some embodiments, the composition or formulation comprising the modified cell or a plurality of the modified cells as described herein may be useful in vaccine production. In some embodiments, the composition or formulation comprising the modified cell or a plurality of the modified cells as described herein may be useful in therapeutics. For example, in some embodiments, the composition or formulation comprising the modified cell or a plurality of the modified cells as described herein may be useful in cellular therapies such as transfusions and transplantations. In other embodiments, a modified T cell expressing a CAR as described herein can be used to treat cancer. In some embodiments, the cancer expresses a protein (e.g., CD19, CD22, BCMA, HER2, IL13Ra2, CD123, FAP, VEGFR-2, ganglioside GD2, EGFRvIII, mesothelin, or EphA) against which the CAR is directed. Accordingly, in some embodiments, the cancer is a myeloma (e.g., multiple myeloma), a lymphoma (e.g., diffuse large B-cell lymphoma (DLBCL), follicular lymphoma, or mantle cell lymphoma), a leukemia (e.g., B-cell acute lymphoblastic leukemia (ALL)), breast cancer, or brain cancer (e.g., glioblastoma).

In some embodiments, the composition or formulation comprising the modified cell or a plurality of the modified cells as described herein may be useful to establish a new cell line comprising a modified genomic sequence. In some embodiments, a modified cell of the disclosure is a modified stem cell (e.g., a modified totipotent/omnipotent stem cell, a modified pluripotent stem cell, a modified multipotent stem cell, a modified oligopotent stem cell, or a modified unipotent stem cell) that differentiates into one or more cell lineages comprising the deletion or insertion of the modified stem cell. The disclosure further provides organisms (such as animals, plants, or fungi) comprising or produced from a modified cell of the disclosure.

### Additional Embodiments

Provided below are additional embodiments, which are also within the scope of the present disclosure.

Embodiment 1: A modified cell comprising a DNA insertion, wherein the DNA insertion is 10 or more nucleotides in length and is adjacent to a 5′-NTTN-3′ sequence, wherein N is any nucleotide.

In any of the modified cells of Embodiment 1, an unmodified cell may lack the DNA insertion.

In any of the modified cells of Embodiment 1, the insertion may be in a genome of the modified cell.

In any of the modified cells of Embodiment 1, the unmodified cell may be a wild-type cell.

In any of the modified cells of Embodiment 1, the insertion may be at least 25, 50, 75, 100, 150, 200, 250, 300, 350, 400, 450, 500, 600, 750, 1000, 1500, 2000, 2500, or 3000 nucleotides in length.

In any of the modified cells of Embodiment 1, the insertion may be downstream of the 5′-NTTN-3′ sequence.

In any of the modified cells of Embodiment 1, the insertion may be within 200 nucleotides downstream of the 5′-NTTN-3′ sequence.

In any of the modified cells of Embodiment 1, the insertion may be upstream of the 5′-NTTN-3′ sequence.

In any of the modified cells of Embodiment 1, the insertion may be within 200 nucleotides downstream of the 5′-NTTN-3′ sequence.

In any of the modified cells of Embodiment 1, the insertion may start within about 3 nucleotides to about 35 nucleotides of the 5′-NTTN-3′ sequence.

In any of the modified cells of Embodiment 1, the insertion may start within about 3 nucleotides upstream of the 5′-NTTN-3′ sequence and about 35 nucleotides downstream of the 5′-NTTN-3′ sequence.

In any of the modified cells of Embodiment 1, the insertion may start within about 3 nucleotides to about 15 nucleotides downstream of the 5′-NTTN-3′ sequence.

In any of the modified cells of Embodiment 1, the insertion may start within about 15 nucleotides to about 30 nucleotides downstream of the 5′-NTTN-3′ sequence.

In any of the modified cells of Embodiment 1, the 5′-NTTN-3′ sequence may be 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G.

In any of the modified cells of Embodiment 1, the 5′-NTTN-3′ sequence may be 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In any of the modified cells of Embodiment 1, the modified cell may be a eukaryotic cell or a prokaryotic cell.

In any of the modified cells of Embodiment 1, the modified cell may be an animal cell, a plant cell, or a fungal cell, or the cell may be derived from an animal cell, a plant cell, or a fungal cell.

In any of the modified cells of Embodiment 1, the modified cell may be a mammalian cell or may be derived from a mammalian cell.

In any of the modified cells of Embodiment 1, the modified cell may be a human cell or may be derived from a human cell.

In any of the modified cells of Embodiment 1, the modified cell may be a stem cell (e.g., a totipotent/omnipotent stem cell, a pluripotent stem cell, a multipotent stem cell, an oligopotent stem cell, or an unipotent stem cell), a differentiated cell, or a terminally differentiated cell.

In any of the modified cells of Embodiment 1, the modified cell may be a primary cell.

In any of the modified cells of Embodiment 1, the modified cell may be from a cell line.

In any of the modified cells of Embodiment 1, the modified cell may be a T cell or may be derived from a T cell.

In any of the modified cells of Embodiment 1, the T cell may be a CD4+ T cell, a CD8+ T cell, or a CD4+/CD8+ T cell.

In any of the modified cells of Embodiment 1, the insertion may be within a gene or a regulatory element thereof.

In any of the modified cells of Embodiment 1, the insertion may be in an exon of a gene.

In any of the modified cells of Embodiment 1, the insertion may overlap with a mutation in a gene.

In any of the modified cells of Embodiment 1, the insertion may overlap with a deletion in a gene.

In any of the modified cells of Embodiment 1, the insertion may inhibit or decreases expression of the gene.

In any of the modified cells of Embodiment 1, the insertion may disrupt one or both alleles of a gene.

In any of the modified cells of Embodiment 1, the insertion may be within a T cell receptor alpha constant (TRAC) gene, a beta-2-microglobulin (B2M) gene, or a regulatory element thereof.

In any of the modified cells of Embodiment 1, the insertion may encode a protein.

In any of the modified cells of Embodiment 1, the insertion may encode a chimeric antigen receptor (CAR), and optionally comprises a regulatory sequence operably linked to the sequence encoding the CAR.

In any of the modified cells of Embodiment 1, the CAR may comprise a single chain antibody (scFv), a transmembrane domain, an intracellular signaling domain, and, optionally, a hinge domain between the scFv and the transmembrane domain.

In any of the modified cells of Embodiment 1, the intracellular signaling domain may comprise a CD3ζ signaling domain.

In any of the modified cells of Embodiment 1, the intracellular signaling domain may further comprise a signaling domain selected from the group consisting of CD28, 4-1BB, ICOS, OX40, CD27, p56-lck, or a combination of two or more thereof.

In any of the modified cells of Embodiment 1, the CAR may comprise an scFv that is specific for CD19, CD22, B cell maturation antigen (BCMA), HER2, IL13Ra2, CD123, fibroblast activation protein (FAP), vascular endothelial growth factor receptor-2 (VEGFR-2), ganglioside GD2, EGFRvIII, mesothelin, or EphA.

In any of the modified cells of Embodiment 1, the cell may be a T cell or may be derived from a T cell, and the insertion may be within the TRAC or B2M locus of the T cell.

Embodiment 2: A modified T cell comprising a DNA insertion, wherein the DNA insertion is 10 or more nucleotides in length and is adjacent to a 5′-NTTN-3′ sequence, wherein N is any nucleotide.

In any of the modified T cells of Embodiment 2, an unmodified T cell may lack the DNA insertion.

In any of the modified T cells of Embodiment 2, the insertion may be in a genome of the modified T cell.

In any of the modified T cells of Embodiment 2, the unmodified T cell may be a wild-type T cell.

In any of the modified T cells of Embodiment 2, the insertion may be at least 25, 50, 75, 100, 150, 200, 250, 300, 350, 400, 450, 500, 600, 750, 1000, 1500, 2000, 2500, or 3000 nucleotides in length.

In any of the modified T cells of Embodiment 2, the insertion may be downstream of the 5′-NTTN-3′ sequence.

In any of the modified T cells of Embodiment 2, the insertion may be within 200 nucleotides downstream of the 5′-NTTN-3′ sequence.

In any of the modified T cells of Embodiment 2, the insertion may be upstream of the 5′-NTTN-3′ sequence.

In any of the modified T cells of Embodiment 2, the insertion may be within 200 nucleotides downstream of the 5′-NTTN-3′ sequence.

In any of the modified T cells of Embodiment 2, the insertion may start within about 3 nucleotides to about 35 nucleotides of the 5′-NTTN-3′ sequence.

In any of the modified T cells of Embodiment 2, the insertion may start within about 3 nucleotides upstream of the 5′-NTTN-3′ sequence and about 35 nucleotides downstream of the 5′-NTTN-3′ sequence.

In any of the modified T cells of Embodiment 2, the insertion may start within about 3 nucleotides to about 15 nucleotides downstream of the 5′-NTTN-3′ sequence.

In any of the modified T cells of Embodiment 2, the insertion may start within about 15 nucleotides to about 30 nucleotides downstream of the 5′-NTTN-3′ sequence.

In any of the modified T cells of Embodiment 2, the 5′-NTTN-3′ sequence may be 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G.

In any of the modified T cells of Embodiment 2, the 5′-NTTN-3′ sequence may be 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In any of the modified T cells of Embodiment 2, the modified T cell may be a human cell or may be derived from a human cell.

In any of the modified T cells of Embodiment 2, the modified T cell may be a primary cell.

In any of the modified T cells of Embodiment 2, the modified T cell may be from a cell line.

In any of the modified T cells of Embodiment 2, the modified T cell may be derived from a T cell.

In any of the modified T cells of Embodiment 2, the modified T cell may be a CD4+ T cell, a CD8+ T cell, or a CD4+/CD8+ T cell.

In any of the modified T cells of Embodiment 2, the insertion may be within a gene or a regulatory element thereof.

In any of the modified T cells of Embodiment 2, the insertion may be in an exon of a gene.

In any of the modified T cells of Embodiment 2, the insertion may overlap with a mutation in a gene.

In any of the modified T cells of Embodiment 2, the insertion may overlap with a deletion in a gene.

In any of the modified T cells of Embodiment 2, the insertion may inhibit or decrease expression of the gene.

In any of the modified T cells of Embodiment 2, the insertion may disrupt one or both alleles of a gene.

In any of the modified T cells of Embodiment 2, the insertion may be within a T cell receptor alpha constant (TRAC) gene, a beta-2-microglobulin (B2M) gene, or a regulatory element thereof.

In any of the modified T cells of Embodiment 2, the insertion may encode a protein.

In any of the modified T cells of Embodiment 2, the insertion may encode a chimeric antigen receptor (CAR), and optionally comprises a regulatory sequence operably linked to the sequence encoding the CAR.

In any of the modified T cells of Embodiment 2, the CAR may comprise a single chain antibody (scFv), a transmembrane domain, an intracellular signaling domain, and, optionally, a hinge domain between the scFv and the transmembrane domain.

In any of the modified T cells of Embodiment 2, the intracellular signaling domain may comprise a CD3ζ signaling domain.

In any of the modified T cells of Embodiment 2, the intracellular signaling domain may further comprise a signaling domain selected from the group consisting of CD28, 4-1BB, ICOS, OX40, CD27, p56-lck, or a combination of two or more thereof.

In any of the modified T cells of Embodiment 2, the CAR may comprise an scFv that is specific for CD19, CD22, B cell maturation antigen (BCMA), HER2, IL13Ra2, CD123, fibroblast activation protein (FAP), vascular endothelial growth factor receptor-2 (VEGFR-2), ganglioside GD2, EGFRvIII, mesothelin, or EphA.

In any of the modified T cells of Embodiment 2, the cell may be a T cell or may be derived from a T cell, and the insertion may be within the TRAC or B2M locus of the T cell.

Embodiment 3: A modified T cell comprising a DNA insertion, wherein the DNA insertion encodes a CAR and is adjacent to a 5′-NTTN-3′ sequence, wherein N is any nucleotide.

In any of the modified cells of Embodiment 3, an unmodified T cell may lack the DNA insertion.

In any of the modified cells of Embodiment 3, the insertion may be in a genome of the modified T cell.

In any of the modified cells of Embodiment 3, the unmodified T cell may be a wild-type T cell.

In any of the modified cells of Embodiment 3, the insertion may be at least 1000, 1500, 2000, 2500, or 3000 nucleotides in length.

In any of the modified cells of Embodiment 3, the insertion may be downstream of the 5′-NTTN-3′ sequence.

In any of the modified cells of Embodiment 3, the insertion may be within 200 nucleotides downstream of the 5′-NTTN-3′ sequence.

In any of the modified cells of Embodiment 3, the insertion may be upstream of the 5′-NTTN-3′ sequence.

In any of the modified cells of Embodiment 3, the insertion may be within 200 nucleotides downstream of the 5′-NTTN-3′ sequence.

In any of the modified cells of Embodiment 3, the insertion may start within about 3 nucleotides to about 35 nucleotides of the 5′-NTTN-3′ sequence.

In any of the modified cells of Embodiment 3, the insertion may start within about 3 nucleotides upstream of the 5′-NTTN-3′ sequence and about 35 nucleotides downstream of the 5′-NTTN-3′ sequence.

In any of the modified cells of Embodiment 3, the insertion may start within about 3 nucleotides to about 15 nucleotides downstream of the 5′-NTTN-3′ sequence.

In any of the modified cells of Embodiment 3, the insertion may start within about 15 nucleotides to about 30 nucleotides downstream of the 5′-NTTN-3′ sequence.

In any of the modified cells of Embodiment 3, the 5′-NTTN-3′ sequence may be 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G.

In any of the modified cells of Embodiment 3, the 5′-NTTN-3′ sequence may be 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In any of the modified cells of Embodiment 3, the modified T cell may be a human cell or may be derived from a human cell.

In any of the modified cells of Embodiment 3, the modified T cell may be a primary cell.

In any of the modified cells of Embodiment 3, the modified T cell may be from a cell line.

In any of the modified cells of Embodiment 3, the modified T cell may be derived from a T cell.

In any of the modified cells of Embodiment 3, the modified T cell may be a CD4+ T cell, a CD8+ T cell, or a CD4+/CD8+ T cell.

In any of the modified cells of Embodiment 3, the insertion may be within a gene or a regulatory element thereof.

In any of the modified cells of Embodiment 3, the insertion may be in an exon of a gene.

In any of the modified cells of Embodiment 3, the insertion may inhibit or decrease expression of the gene.

In any of the modified cells of Embodiment 3, the insertion may disrupt one or both alleles of a gene.

In any of the modified cells of Embodiment 3, the insertion may be within a T cell receptor alpha constant (TRAC) gene, a beta-2-microglobulin (B2M) gene, or a regulatory element thereof.

In any of the modified cells of Embodiment 3, the insertion may comprise a regulatory sequence operably linked to the sequence encoding the CAR.

In any of the modified cells of Embodiment 3, the CAR may comprise a single chain antibody (scFv), a transmembrane domain, an intracellular signaling domain, and, optionally, a hinge domain between the scFv and the transmembrane domain.

In any of the modified cells of Embodiment 3, the intracellular signaling domain may comprise a CD3ζ signaling domain.

In any of the modified cells of Embodiment 3, the intracellular signaling domain may further comprise a signaling domain selected from the group consisting of CD28, 4-1BB, ICOS, OX40, CD27, p56-lck, or a combination of two or more thereof.

In any of the modified cells of Embodiment 3, the CAR may comprise an scFv that is specific for CD19, CD22, B cell maturation antigen (BCMA), HER2, IL13Ra2, CD123, fibroblast activation protein (FAP), vascular endothelial growth factor receptor-2 (VEGFR-2), ganglioside GD2, EGFRvIII, mesothelin, or EphA.

Embodiment 4: Progeny of the modified cell or modified T cell of any one of the previous Embodiments.

Embodiment 5: A method of obtaining a plurality of modified cells of any one of the previous Embodiments, wherein the method comprises culturing the modified cell of any one of the previous Embodiments.

Embodiment 6: A method of obtaining a plurality of modified cells of any one of the previous Embodiments, wherein the method comprises isolating and culturing the modified cell of any one of the previous Embodiments.

Embodiment 7: A plurality of cells, wherein at least 50% of the cells comprise an insertion in a gene, wherein the insertion is at least 10 nucleotides in length and is adjacent to a 5′-NTTN-3′ sequence, wherein N is any nucleotide.

In any of the plurality of cells of Embodiment 7, at least about 55%, 60%, 65%, 70%, 75%, 80%, 85%, 90%, 95%, or 99% of the cells may comprise the insertion.

In any of the plurality of cells of Embodiment 7, 100% of the cells may comprise the insertion.

In any of the plurality of cells of Embodiment 7, the insertion may be at least 25, 50, 75, 100, 150, 200, 250, 300, 350, 400, 450, 500, 600, 750, 1000, 1500, 2000, 2500, or 3000 nucleotides in length.

In any of the plurality of cells of Embodiment 7, the insertion may be downstream of the 5′-NTTN-3′ sequence.

In any of the plurality of cells of Embodiment 7, the insertion may be within 200 nucleotides downstream of the 5′-NTTN-3′.

In any of the plurality of cells of Embodiment 7, the insertion may be upstream of the 5′-NTTN-3′ sequence.

In any of the plurality of cells of Embodiment 7, the insertion may be within 200 nucleotides upstream of the 5′-NTTN-3′.

In any of the plurality of cells of Embodiment 7, the insertion may start within about 3 nucleotides to about 35 nucleotides of the 5′-NTTN-3′ sequence.

In any of the plurality of cells of Embodiment 7, the insertion may start within about 3 nucleotides upstream of the 5′-NTTN-3′ sequence and about 35 nucleotides downstream of the 5′-NTTN-3′ sequence.

In any of the plurality of cells of Embodiment 7, the insertion may start within about 3 nucleotides to about 15 nucleotides downstream of the 5′-NTTN-3′ sequence.

In any of the plurality of cells of Embodiment 7, the insertion may start within about 15 nucleotides to about 30 nucleotides downstream of the 5′-NTTN-3′ sequence.

In any of the plurality of cells of Embodiment 7, the 5′-NTTN-3′ sequence may be 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G.

In any of the plurality of cells of Embodiment 7, the 5′-NTTN-3′ sequence may be 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In any of the plurality of cells of Embodiment 7, the plurality of cells may be eukaryotic cells or prokaryotic cells.

In any of the plurality of cells of Embodiment 7, the plurality of cells may be animal cells, plant cells, or fungal cells or the cells derived from animal cells, plant cells, or fungal cells.

In any of the plurality of cells of Embodiment 7, the plurality of cells may be mammalian cells or may be derived from mammalian cells.

In any of the plurality of cells of Embodiment 7, the plurality of cells may be human cells or may be derived from human cells.

In any of the plurality of cells of Embodiment 7, the plurality of cells may be stem cells (e.g., totipotent/omnipotent stem cells, pluripotent stem cells, multipotent stem cells, oligopotent stem cells, or unipotent stem cells), differentiated cells, or terminally differentiated cells.

In any of the plurality of cells of Embodiment 7, the plurality of cells may be primary cells.

In any of the plurality of cells of Embodiment 7, the plurality of cells may be cells of a cell line.

In any of the plurality of cells of Embodiment 7, the plurality of cells may be T cells or may be derived from T cells.

In any of the plurality of cells of Embodiment 7, the plurality of cells may be a co-culture of two or more cell types.

In any of the plurality of cells of Embodiment 7, the insertion may be within a gene or a regulatory element thereof.

In any of the plurality of cells of Embodiment 7, the insertion may be in an exon of a gene.

In any of the plurality of cells of Embodiment 7, the insertion may inhibit or decrease expression of the gene.

In any of the plurality of cells of Embodiment 7, the insertion may disrupt one or both alleles of a gene.

In any of the plurality of cells of Embodiment 7, the insertion may be within a TRAC gene, a B2M gene, or a regulatory element thereof.

In any of the plurality of cells of Embodiment 7, the insertion may encode a protein.

In any of the plurality of cells of Embodiment 7, the insertion may encode a CAR, and optionally comprises a regulatory sequence operably linked to the sequence encoding the CAR.

In any of the plurality of cells of Embodiment 7, the CAR may comprise an scFv, a transmembrane domain, an intracellular signaling domain, and, optionally, a hinge domain between the scFv and the transmembrane domain.

In any of the plurality of cells of Embodiment 7, the intracellular signaling domain may comprise a CD3ζ signaling domain.

In any of the plurality of cells of Embodiment 7, the intracellular signaling domain further may comprise a signaling domain selected from the group consisting of CD28, 4-1BB, ICOS, OX40, CD27, p56-lck, or a combination of two or more thereof.

In any of the plurality of cells of Embodiment 7, the CAR may be specific for CD19, CD22, BCMA, HER2, IL13Ra2, CD123, FAP, VEGFR-2, ganglioside GD2, EGFRvIII, mesothelin, or EphA.

In any of the plurality of cells of Embodiment 7, the cell may be a T cell or may be derived from a T cell, and the insertion may be within the TRAC or B2M locus of the T cell.

In any of the plurality of cells of Embodiment 7, the T cell may be a CD4+ T cell, a CD8+ T cell, or a CD4+/CD8+ T cell.

Embodiment 8: A plurality of T cells, wherein at least 50% of the cells comprise an insertion in a gene, wherein the insertion encodes a CAR and is adjacent to a 5′-NTTN-3′ sequence, wherein N is any nucleotide.

In any of the plurality of cells of any of the Embodiments herein, at least about 55%, 60%, 65%, 70%, 75%, 80%, 85%, 90%, 95%, or 99% of the cells may comprise the insertion.

In any of the plurality of cells of any of the Embodiments herein, 100% of the cells may comprise the insertion.

In any of the plurality of cells of any of the Embodiments herein, the insertion may be at least 1000, 1500, 2000, 2500, or 3000 nucleotides in length.

In any of the plurality of cells of any of the Embodiments herein, the insertion may be downstream of the 5′-NTTN-3′ sequence.

In any of the plurality of cells of any of the Embodiments herein, the insertion may be within 200 nucleotides downstream of the 5′-NTTN-3′.

In any of the plurality of cells of any of the Embodiments herein, the insertion may be upstream of the 5′-NTTN-3′ sequence.

In any of the plurality of cells of any of the Embodiments herein, the insertion may be within 200 nucleotides upstream of the 5′-NTTN-3′.

In any of the plurality of cells of any of the Embodiments herein, the insertion may start within about 3 nucleotides to about 35 nucleotides of the 5′-NTTN-3′ sequence.

In any of the plurality of cells of any of the Embodiments herein, the insertion may start within about 3 nucleotides upstream of the 5′-NTTN-3′ sequence and about 35 nucleotides downstream of the 5′-NTTN-3′ sequence.

In any of the plurality of cells of any of the Embodiments herein, the insertion may start within about 15 nucleotides to about 30 nucleotides downstream of the 5′-NTTN-3′ sequence.

In any of the plurality of cells of any of the Embodiments herein, the insertion may start within about 3 nucleotides to about 15 nucleotides downstream of the 5′-NTTN-3′ sequence.

In any of the plurality of cells of any of the Embodiments herein, the 5′-NTTN-3′ sequence may be 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G.

In any of the plurality of cells of any of the Embodiments herein, the 5′-NTTN-3′ sequence may be 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In any of the plurality of cells of any of the Embodiments herein, the T cells may be human cells or may be derived from human cells.

In any of the plurality of cells of any of the Embodiments herein, the T cells may be primary cells.

In any of the plurality of cells of any of the Embodiments herein, the T cells may be from a cell line.

In any of the plurality of cells of any of the Embodiments herein, the T cells may be derived from a T cell.

In any of the plurality of cells of any of the Embodiments herein, the T cells may be CD4+ T cells, CD8+ T cells, or CD4+/CD8+ T cells.

In any of the plurality of cells of any of the Embodiments herein, the T cells may be CD4+ T cells and CD8+ T cells at a ratio of about 1:1.

In any of the plurality of cells of any of the Embodiments herein, the insertion may be within a gene or a regulatory element thereof.

In any of the plurality of cells of any of the Embodiments herein, the insertion may be in an exon of a gene.

In any of the plurality of cells of any of the Embodiments herein, the insertion may inhibit or decrease expression of the gene.

In any of the plurality of cells of any of the Embodiments herein, the insertion may disrupt one or both alleles of a gene.

In any of the plurality of cells of any of the Embodiments herein, the insertion may be within a T cell receptor alpha constant (TRAC) gene, a beta-2-microglobulin (B2M) gene, or a regulatory element thereof.

In any of the plurality of cells of any of the Embodiments herein, the insertion may comprise a regulatory sequence operably linked to the sequence encoding the CAR.

In any of the plurality of cells of any of the Embodiments herein, the CAR may comprise a single chain antibody (scFv), a transmembrane domain, an intracellular signaling domain, and, optionally, a hinge domain between the scFv and the transmembrane domain.

In any of the plurality of cells of any of the Embodiments herein, the intracellular signaling domain may comprise a CD3ζ signaling domain.

In any of the plurality of cells of any of the Embodiments herein, the intracellular signaling domain may further comprise a signaling domain selected from the group consisting of CD28, 4-1BB, ICOS, OX40, CD27, p56-lck, or a combination of two or more thereof.

In any of the plurality of cells of any of the Embodiments herein, the CAR may comprise an scFv that is specific for CD19, CD22, B cell maturation antigen (BCMA), HER2, IL13Ra2, CD123, fibroblast activation protein (FAP), vascular endothelial growth factor receptor-2 (VEGFR-2), ganglioside GD2, EGFRvIII, mesothelin, or EphA.

Embodiment 8: A composition or formulation comprising a modified cell or a plurality of cells comprising an insertion, wherein the insertion is 10 or more nucleotides in length and is adjacent to a 5′-NTTN-3′ sequence, wherein N is any nucleotide.

In any of the compositions or formulations of Embodiment 8, at least about 50% of the cells may comprise the insertion.

In any of the compositions or formulations of Embodiment 8, at least about 55%, 60%, 65%, 70%, 75%, 80%, 85%, 90%, 95%, or 99% of the cells may be comprise the insertion.

In any of the compositions or formulations of Embodiment 8, 100% of the cells may comprise the insertion.

In any of the compositions or formulations of Embodiment 8, the insertion may be at least 25, 50, 75, 100, 150, 200, 250, 300, 350, 400, 450, 500, 600, 750, 1000, 1500, 2000, 2500, or 3000 nucleotides in length.

In any of the compositions or formulations of Embodiment 8, the insertion may be downstream of the 5′-NTTN-3′ sequence.

In any of the compositions or formulations of Embodiment 8, the insertion may be within 200 nucleotides downstream of the 5′-NTTN-3′.

In any of the compositions or formulations of Embodiment 8, the insertion may be upstream of the 5′-NTTN-3′ sequence.

In any of the compositions or formulations of Embodiment 8, the insertion may be within 200 nucleotides downstream of the 5′-NTTN-3′.

In any of the compositions or formulations of Embodiment 8, the insertion may start within about 3 nucleotides to about 35 nucleotides of the 5′-NTTN-3′ sequence.

In any of the compositions or formulations of Embodiment 8, the insertion may start within about 3 nucleotides upstream of the 5′-NTTN-3′ sequence and about 35 nucleotides downstream of the 5′-NTTN-3′ sequence.

In any of the compositions or formulations of Embodiment 8, the insertion may start within about 15 nucleotides to about 30 nucleotides downstream of the 5′-NTTN-3′ sequence.

In any of the compositions or formulations of Embodiment 8, the insertion may start within about 3 nucleotides to about 15 nucleotides downstream of the 5′-NTTN-3′ sequence.

In any of the compositions or formulations of Embodiment 8, the 5′-NTTN-3′ sequence may be 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G.

In any of the compositions or formulations of Embodiment 8, the 5′-NTTN-3′ sequence may be 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In any of the compositions or formulations of Embodiment 8, the cell may be a eukaryotic cell or a prokaryotic cell.

In any of the compositions or formulations of Embodiment 8, the modified cell or a cell of the plurality of modified cells may be an animal cell, a plant cell, or a fungal cell or the cell may be derived from an animal cell, a plant cell, or a fungal cell.

In any of the compositions or formulations of Embodiment 8, the modified cell or a cell of the plurality of modified cells may be a mammalian cell or may be derived from a mammalian cell.

In any of the compositions or formulations of Embodiment 8, the modified cell or a cell of the plurality of modified cells may be a human cell or may be derived from a human cell.

In any of the compositions or formulations of Embodiment 8, the modified cell or a cell of the plurality of modified cells may be a stem cell (e.g., a totipotent/omnipotent stem cell, a pluripotent stem cell, a multipotent stem cell, an oligopotent stem cell, or an unipotent stem cell), a differentiated cell, or a terminally differentiated cell.

In any of the compositions or formulations of Embodiment 8, the modified cell or a cell of the plurality of modified cells may be a primary cell.

In any of the compositions or formulations of Embodiment 8, the modified cell or a cell of the plurality of modified cells may be a cell from a cell line.

In any of the compositions or formulations of Embodiment 8, the modified cell or a cell of the plurality of modified cells may be a T cell or may be derived from a T cell.

In any of the compositions or formulations of Embodiment 8, the T cell may be a CD4+ T cell, a CD8+ T cell, or a CD4+/CD8+ T cell.

In any of the compositions or formulations of Embodiment 8, the insertion may be within a gene or a regulatory element thereof.

In any of the compositions or formulations of Embodiment 8, the insertion may be in an exon of a gene.

In any of the compositions or formulations of Embodiment 8, the insertion may inhibit or decrease expression of the gene.

In any of the compositions or formulations of Embodiment 8, the insertion may disrupt one or both alleles of a gene.

In any of the compositions or formulations of Embodiment 8, the insertion may be within a TRAC gene, a B2M gene, or a regulatory element thereof.

In any of the compositions or formulations of Embodiment 8, the insertion may encode a protein.

In any of the compositions or formulations of Embodiment 8, the insertion may encode a CAR, and optionally may comprise a regulatory sequence operably linked to the sequence encoding the CAR.

In any of the compositions or formulations of Embodiment 8, the CAR may comprise an scFv, a transmembrane domain, an intracellular signaling domain, and, optionally, a hinge domain between the scFv and the transmembrane domain.

In any of the compositions or formulations of Embodiment 8, the intracellular signaling domain may comprise a CD3ζ signaling domain.

In any of the compositions or formulations of Embodiment 8, the intracellular signaling domain may further comprise a signaling domain selected from the group consisting of CD28, 4-1BB, ICOS, OX40, CD27, p56-lck, or a combination of two or more thereof.

In any of the compositions or formulations of Embodiment 8, the CAR may comprise an scFv that is specific for CD19, CD22, BCMA, HER2, IL13Ra2, CD123, FAP, VEGFR-2, ganglioside GD2, EGFRvIII, mesothelin, or EphA.

In any of the modified cells of any of the Embodiments described herein, the cell may be a T cell or may be derived from a T cell, and the insertion may be within the TRAC or B2M locus of the T cell.

Embodiment 9: A composition or formulation comprising a modified T cell or a plurality of T cells comprising an insertion, wherein the insertion encodes a CAR and is adjacent to a 5′-NTTN-3′ sequence, wherein N is any nucleotide.

In any of the compositions or formulations of Embodiment 9, at least about 50% of the cells may comprise the insertion.

In any of the compositions or formulations of Embodiment 9, at least about 55%, 60%, 65%, 70%, 75%, 80%, 85%, 90%, 95%, or 99% of the cells may comprise the insertion.

In any of the compositions or formulations of Embodiment 9, 100% of the cells may comprise the insertion.

In any of the compositions or formulations of Embodiment 9, the insertion may be at least 1000, 1500, 2000, 2500, or 3000 nucleotides in length.

In any of the compositions or formulations of Embodiment 9, the insertion may be downstream of the 5′-NTTN-3′ sequence.

In any of the compositions or formulations of Embodiment 9, the insertion may be within 200 nucleotides downstream of the 5′-NTTN-3′.

In any of the compositions or formulations of Embodiment 9, the insertion may be upstream of the 5′-NTTN-3′ sequence.

In any of the compositions or formulations of Embodiment 9, the insertion may be within 200 nucleotides upstream of the 5′-NTTN-3′.

In any of the compositions or formulations of Embodiment 9, the insertion may start within about 3 nucleotides to about 35 nucleotides of the 5′-NTTN-3′ sequence.

In any of the compositions or formulations of Embodiment 9, the insertion may start within about 3 nucleotides upstream of the 5′-NTTN-3′ sequence and about 35 nucleotides downstream of the 5′-NTTN-3′ sequence.

In any of the compositions or formulations of Embodiment 9, the insertion may start within about 15 nucleotides to about 30 nucleotides downstream of the 5′-NTTN-3′ sequence.

In any of the compositions or formulations of Embodiment 9, the insertion may start within about 3 nucleotides to about 15 nucleotides downstream of the 5′-NTTN-3′ sequence.

In any of the compositions or formulations of Embodiment 9, the 5′-NTTN-3′ sequence may be 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G.

In any of the compositions or formulations of Embodiment 9, the 5′-NTTN-3′ sequence may be 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In any of the compositions or formulations of Embodiment 9, the T cells may be human cells or may be derived from human cells.

In any of the compositions or formulations of Embodiment 9, the T cells may be primary cells.

In any of the compositions or formulations of Embodiment 9, the T cells may be from a cell line.

In any of the compositions or formulations of Embodiment 9, the T cells may be derived from a T cell.

In any of the compositions or formulations of Embodiment 9, T cells may be CD4+ T cells, CD8+ T cells, or CD4+/CD8+ T cells.

In any of the compositions or formulations of Embodiment 9, the insertion may be within a gene or a regulatory element thereof.

In any of the compositions or formulations of Embodiment 9, the insertion may be in an exon of a gene.

In any of the compositions or formulations of Embodiment 9, the insertion may inhibit or decrease expression of the gene.

In any of the compositions or formulations of Embodiment 9, insertion may disrupt one or both alleles of a gene.

In any of the compositions or formulations of Embodiment 9, the insertion may be within a T cell receptor alpha constant (TRAC) gene, a beta-2-microglobulin (B2M) gene, or a regulatory element thereof.

In any of the compositions or formulations of Embodiment 9, the insertion may comprise a regulatory sequence operably linked to the sequence encoding the CAR.

In any of the compositions or formulations of Embodiment 9, the CAR may comprise a single chain antibody (scFv), a transmembrane domain, an intracellular signaling domain, and, optionally, a hinge domain between the scFv and the transmembrane domain.

In any of the compositions or formulations of Embodiment 9, the intracellular signaling domain may comprise a CD3ζ signaling domain.

In any of the compositions or formulations of Embodiment 9, the intracellular signaling domain may further comprise a signaling domain selected from the group consisting of CD28, 4-1BB, ICOS, OX40, CD27, p56-lck, or a combination of two or more thereof.

In any of the compositions or formulations of Embodiment 9, the CAR may comprise an scFv that is specific for CD19, CD22, B cell maturation antigen (BCMA), HER2, IL13Ra2, CD123, fibroblast activation protein (FAP), vascular endothelial growth factor receptor-2 (VEGFR-2), ganglioside GD2, EGFRvIII, mesothelin, or EphA.

### Examples

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—HDR-Mediated Integration of a CAR at the TRAC Locus of a T Cell Using Cas12i

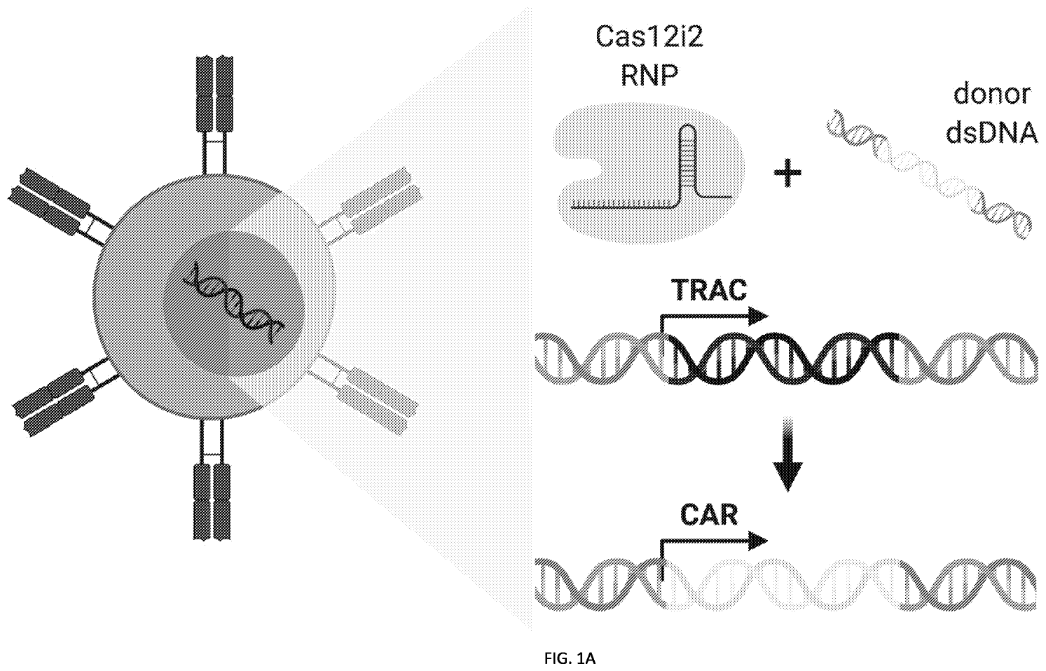

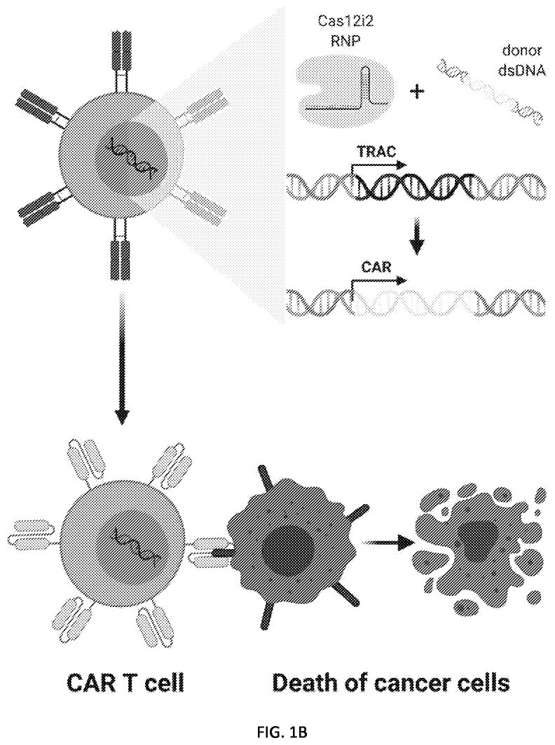

This Example describes generation of a T cell harboring a chimeric antigen receptor (CAR) at the TRAC locus using site-specific HDR knockin with a Cas12i RNP. See FIG. 1A.

T cells are electroporated with a Cas12i protein complexed to an RNA guide designed to disrupt the TRAC locus. The RNA guide is precomplexed on ice with a Cas12i polypeptide at a 2:1 molar ratio to generate a TRAC RNP. An exemplary RNA guide sequence is a Cas12i RNA guide having the following sequence:

AGAAAUCCGUCUUUCAUUGACGGAGAGTCTCTCAGCTGGTACA.

Subsequently, the electroporated T cells are transduced with an adeno-associated virus (AAV) vector that supplies the CAR cDNA and homology arms flanking the cut site. The AAV serves as the template for HDR repair and by extension, precision integration of the CAR at the TRAC locus. A schematic of a representative vector is shown in FIG. 2A. The linkers preceding and following T2A are optional. Any linkers that do not interfere with T2A can be used. A stop codon follows the CAR, which is shown in FIG. 2B.

The following sequence is from a recombinant TRAC-1928z AAV6 vector comprising a self-cleaving P2A peptide in frame with the first exon of TRAC followed by the 1928z CAR 1.9 kb and homology arms flanking the RNA guide targeting sequence:

aggtttcctt gagtggcagg ccaggcctgg ccgtgaacgt tcactgaaat catggcctct
tggccaagat tgatagcttg tgcctgtccc tgagtcccag tccatcacga gcagctggtt
tctaagatgc tatttcccgt ataaagcatg agaccgtgac ttgccagccc cacagagccc
cgcccttgtc catcactggc atctggactc cagcctgggt tggggcaaag agggaaatga
gatcatgtcc taaccctgat cctcttgtcc cacagatatc cagaaccctg accctgccgt
gtacagtggc tccggtgccc gtcagtgggc agagcgcaca tcgcccacag tccccgagaa
gttgggggga ggggtcggca attgaaccgg tgcctagaga aggtggcgcg gggtaaactg
ggaaagtgat gtcgtgtact ggctccgcct ttttcccgag ggtgggggag aaccgtatat
aagtgcagta gtcgccgtga acgttctttt tcgcaacggg tttgccgcca gaacacaggt
gtcgtgacgc gggatccgcc accatgctca ggctgctctt ggctctcaac ttattccctt
caattcaagt aacaggaggg tcttcggact acaaggatca tgacggagac tataaggatc
acgatattga ttacaaagat gacgacgaca aagacatcca gatgacacag actacatcct
ccctgtctgc ctctctggga gacagagtca ccatcagttg cagggcaagt caggacatct
ctaagtattt gaattggtat cagcagaaac cagatggaac tgttaaactc ctgatctacc
atacatcaag attacactca ggagtcccat caaggttcag tggcagtggg tctggaacag
attattctct caccattagc aacctggagc aagaagatat tgccacttac ttttgccaac
agggtaatac gcttccgtac acgttcggag gggggactaa gttggaaata acaggctcca
cctctggatc cggcaagccc ggatctggcg agggatccac caagggcgag gtgaaactgc
aggagtcagg acctggcctg gtggcgccct cacagagcct gtccgtcaca tgcactgtct
caggggtctc attacccgac tatggtgtaa gctggattcg ccagcctcca cgaaagggtc
tggagtggct gggagtaata tggggtagtg aaaccacata ctataattca gctctcaaat
ccagactgac catcatcaag gacaactcca agagccaagt tttcttaaaa atgaacagtc
tgcaaactga tgacacagcc atttactact gtgccaaaca ttattactac ggtggtagct
atgctatgga ctactggggt caaggaacct cagtcaccgt ctcctcagcg gccgcaggta
ccaccacaac gcccgctcct cggccaccga cgccagcgcc aactattgcg agtcagcctc
tcagtctgcg acctgaggct tgtcgaccag cagccggagg cgcagtgcac acgagggggc
tggacttcgc ctgtgataga agacctcctt ctaagccctt ttgggtgctg gtggtggttg
gtggagtcct ggcttgctat agcttgctag taacagtggc ctttattatt ttctgggtga
ggaaacgggg cagaaagaaa ctcctgtata tattcaaaca accatttatg agaccagtac
aaactactca agaggaagat ggctgtagct gccgatttcc agaagaagaa gaaggaggat
gtgaactggc tagcctgaga gtgaagttca gcaggagcgc agacgccccc gcgtaccagc
agggccagaa ccagctctat aacgagctca atctaggacg aagagaggag tacgatgttt
tggacaagag acgtggccgg gaccctgaga tggggggaaa gccgcagaga aggaagaacc
ctcaggaagg cctgtacaat gaactgcaga aagataagat ggcggaggcc tacagtgaga
ttgggatgaa aggcgagcgc cggaggggca aggggcacga tggcctttac cagggtctca
gtacagccac caaggacacc tacgacgccc ttcacatgca ggccctgccc cctcgcgcta
gctaacagag agtgagagga acttgtttat tgcagcttat aatggttaca aataaagcaa
tagcatcaca aatttcacaa ataaagcatt tttttcactg cattctagtt gtggtttgtc
caaactcatc aatgtatctt atcatgtctg gctctagcta tcccgtggcc attcctgaag
caaggaaaca gcctgcgaag gcaccaaagc tgcccttacc tgggctgggg aagaaggtgt
cttctggaat aatgctgttg ttgaaggcgt ttgcacatgc aaagtcagat ttgttgctcc
aggccacagc actgttgctc ttgaagtcca tagacctcat gtctagcaca gttttgtctg
tgatatacac atcagaatcc ttactttgtg acacatttgt ttgagaatca aaatcggtga
ataggcagac agacttgtca ctggatttag agtctctcag ctgcgagatt tttgcggccg
caggaacccc tagtgatgga gttggccact ccctctctgc gcgctcgctc gctcactgag
gccgggcgac caaaggtcgc ccgacgcccg ggctttgccc gggcggcctc agtgagcgag
cgagcgcgca gctgcctgca ggggcgcctg atgcggtatt ttctccttac gcatctgtgc
ggtatttcac accgcatacg tcaaagcaac catagtacgc gccctgtagc ggcgcattaa
gcgcggcggg tgtggtggtt acgcgcagcg tgaccgctac acttgccagc gccttagcgc
ccgctccttt cgctttcttc ccttcctttc tcgccacgtt cgccggcttt ccccgtcaag
ctctaaatcg ggggctccct ttagggttcc gatttagtgc tttacggcac ctcgacccca
aaaaacttga tttgggtgat ggttcacgta gtgggccatc gccctgatag acggtttttc
gccctttgac gttggagtcc acgttcttta atagtggact cttgttccaa actggaacaa
cactcaactc tatctcgggc tattcttttg atttataagg gattttgccg atttcggtct
attggttaaa aaatgagctg atttaacaaa aatttaacgc gaattttaac aaaatattaa
cgtttacaat tttatggtgc actctcagta caatctgctc tgatgccgca tagttaagcc
agccccgaca cccgccaaca cccgctgacg cgccctgacg ggcttgtctg ctcccggcat
ccgcttacag acaagctgtg accgtctccg ggagctgcat gtgtcagagg ttttcaccgt
catcaccgaa acgcgcgaga cgaaagggcc tcgtgatacg cctattttta taggttaatg
tcatgataat aatggtttct tagacgtcag gtggcacttt tcggggaaat gtgcgcggaa
cccctatttg tttatttttc taaatacatt caaatatgta tccgctcatg agacaataac
cctgataaat gcttcaataa tattgaaaaa ggaagagtat gagtattcaa catttccgtg
tcgcccttat tccctttttt gcggcatttt gccttcctgt ttttgctcac ccagaaacgc
tggtgaaagt aaaagatgct gaagatcagt tgggtgcacg agtgggttac atcgaactgg
atctcaacag cggtaagatc cttgagagtt ttcgccccga agaacgtttt ccaatgatga
gcacttttaa agttctgcta tgtggcgcgg tattatcccg tattgacgcc gggcaagagc
aactcggtcg ccgcatacac tattctcaga atgacttggt tgagtactca ccagtcacag
aaaagcatct tacggatggc atgacagtaa gagaattatg cagtgctgcc ataaccatga
gtgataacac tgcggccaac ttacttctga caacgatcgg aggaccgaag gagctaaccg
cttttttgca caacatgggg gatcatgtaa ctcgccttga tcgttgggaa ccggagctga
atgaagccat accaaacgac gagcgtgaca ccacgatgcc tgtagcaatg gcaacaacgt
tgcgcaaact attaactggc gaactactta ctctagcttc ccggcaacaa ttaatagact
ggatggaggc ggataaagtt gcaggaccac ttctgcgctc ggcccttccg gctggctggt
ttattgctga taaatctgga gccggtgagc gtggaagccg cggtatcatt gcagcactgg
ggccagatgg taagccctcc cgtatcgtag ttatctacac gacggggagt caggcaacta
tggatgaacg aaatagacag atcgctgaga taggtgcctc actgattaag cattggtaac
tgtcagacca agtttactca tatatacttt agattgattt aaaacttcat ttttaattta
aaaggatcta ggtgaagatc ctttttgata atctcatgac caaaatccct taacgtgagt
tttcgttcca ctgagcgtca gaccccgtag aaaagatcaa aggatcttct tgagatcctt
tttttctgcg cgtaatctgc tgcttgcaaa caaaaaaacc accgctacca gcggtggttt
gtttgccgga tcaagagcta ccaactcttt ttccgaaggt aactggcttc agcagagcgc
agataccaaa tactgttctt ctagtgtagc cgtagttagg ccaccacttc aagaactctg
tagcaccgcc tacatacctc gctctgctaa tcctgttacc agtggctgct gccagtggcg
ataagtcgtg tcttaccggg ttggactcaa gacgatagtt accggataag gcgcagcggt
cgggctgaac ggggggttcq tgcacacagc ccagcttgga gcgaacgacc tacacciaac
tgagatacct acagcgtgag ctatgagaaa gcgccacgct tcccgaaggg agaaaggcgg
acaggtatcc ggtaagcggc agggtcggaa caggagagcg cacgagggag cttccagggg
gaaacgcctg gtatctttat agtcctgtcg ggtttcgcca cctctgactt gagcgtcgat
ttttgtgatg ctcgtcaggg gggcggagcc tatggaaaaa cgccagcaac gcggcctttt
tacggttcct ggccttttgc tggccttttg ctcacatgtc ctgcaggcag ctgcgcgctc
gctcgctcac tgaggccgcc cgggcgtcgg gcgacctttg gtcgcccggc ctcagtgagc
gagcgagcgc gcagagaggg agtggccaac tccatcacta ggggttcctg cggcctctag
actcgaggcg ttgaca

The Cas12i RNP is delivered to T cells that have been stimulated for 2-3 days with CD3/CD28. Approximately 2-4 hours post electroporation, the T cells are transduced with recombinant TRAC-1928z AAV6 at a multiplicity of infection (MOI) of 1×10⁵-1×10⁷. T cells are cultured and expanded in T-cell growth medium and maintained at a density of ˜1×10⁶ cells per ml. FACS analysis is used to quantify CAR 40 positive, TRAC negative cells to confirm targeting of the CAR into the TRAC locus approximately 4 days post TRAC targeting. Additional confirmation of TRAC targeting specificity includes mapping AAV vector integration over the whole genome.

The TRAC-CAR T cells can undergo functional testing to assess tumor cell killing potency and cytotoxicity using in vitro assays such as tumor cell co-cultures. In such studies, tumor cells will be cultured with the TRAC-CART cells at varying tumor cell to TRAC-CART cell ratios. After co-culture, assays such as enzyme-linked immunospot technique (ELISPOT) or enzyme-linked immunosorbent assay (ELISA) may be used to measure cytokine secretion levels, including interferon-gamma, as a proxy for engineered T cell function. Functionality may also be assessed using in vivo assays in which the engineered TRAC-CART cells are adoptively transferred to mice harboring CAR positive tumors. TRAC-CART functionality will be assessed by their ability to reduce tumor burden and enhance survival. See FIG. 1B.

Therefore, this Example describes engineering of a T cell with enhanced potency for tumor cell killing.

What is claimed is:

1. A method of making a modified T cell, the method comprising introducing into a T cell:
(a) a variant Cas12i polypeptide or a nucleic acid encoding the variant Cas12i polypeptide,
(b) a first RNA guide or a nucleic acid encoding the first RNA guide, and
(c) an exogenous nucleic acid for integration into the genome of the T cell.

2. A method of making a modified T cell, the method comprising:
(a) disrupting a gene in the genome of a T cell using a variant Cas12i polypeptide and a first RNA guide, and
(b) introducing an exogenous nucleic acid into the genome of the T cell.

3. The method of claim 1 or 2, wherein the variant Cas12i polypeptide is a variant Cas12i2 polypeptide.

4. The method of any one of claims 1 to 3, wherein the variant Cas12i polypeptide comprises a sequence having at least 90% identity to a sequence of any one of SEQ ID NOs: 3-7.

5. The method of any one of claims 1 to 4, wherein the variant Cas12i polypeptide comprises a sequence of any one of SEQ ID NOs: 3-7.

6. The method of any one of claims 1 to 5, wherein the integration of the exogenous nucleic acid is within 100 nucleotides of a 5′-NTTN-3′ sequence.

7. The method of any one of claims 1 to 6, wherein the first RNA guide comprises a spacer sequence specific to a TRAC gene or to a B2M gene.

8. The method of claim 7, wherein the TRAC gene or the B2M gene is disrupted.

9. The method of claim 8, wherein the disruption is a deletion.

10. The method of claim 8, wherein the disruption is an insertion.

11. The method of any one of claims 8 to 10, wherein the disruption occurs in one or both alleles of the TRAC gene or the B2M gene.

12. The method of any one of claims 8 to 11, wherein the disruption inhibits or decreases expression of the TRAC gene or the B2M gene.

13. The method of any one of claims 8 to 12, wherein the disruption is within 100 nucleotides of a 5′-NTTN-3′ sequence.

14. The method of any one of claims 2 to 13, wherein the exogenous nucleic acid is integrated into the genome.

15. The method of claim 14, wherein the exogenous nucleic acid is integrated into the TRAC gene or the B2M gene.

16. The method of any one of claims 1 to 15, wherein the exogenous nucleic acid comprises a sequence encoding a protein.

17. The method of any one of claims 1 to 16, wherein the exogenous nucleic acid comprises a regulatory sequence operably linked to the sequence encoding the protein.

18. The method of claim 16 or 17, wherein the protein is a chimeric antigen receptor (CAR).

19. The method of claim 18, wherein the CAR comprises a single chain antibody (scFv), a transmembrane domain, an intracellular signaling domain, and, optionally, a hinge domain between the scFv and the transmembrane domain.

20. The method of claim 19, wherein the intracellular signaling domain comprises a CD3ζ signaling domain.

21. The method of claim 19 or 20, wherein the intracellular signaling domain further comprises a signaling domain selected from the group consisting of CD28, 4-1BB, ICOS, OX40, CD27, p56-lck, or a combination of two or more thereof.

22. The method of any one of claims 18 to 21, wherein the CAR comprises a scFv that is specific for CD19, CD22, BCMA, HER2, IL13Ra2, CD123, FAP, VEGFR-2, ganglioside GD2, EGFRvIII, mesothelin, or EphA.

23. The method of any one of claims 1 to 22, wherein the exogenous nucleic acid is integrated into the genome of the T cell by homology directed repair (HDR).

24. The method of any one of claims 1 to 23, wherein the exogenous nucleic acid is included in a vector.

25. The method of claim 24, wherein the vector is a viral vector.

26. The method of claim 25, wherein the viral vector is a lentiviral vector or an AAV vector.

27. The method of any one of claims 1 to 26, wherein the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the TRAC gene.

28. The method of any one of claims 1 to 26, wherein the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the B2M gene.

29. A modified T cell generated by a method of any one of claims 1 to 28.

30. The modified T cell of claim 29, which is a human T cell or is derived from a human cell or a human T cell.

31. A composition or formulation comprising one or more modified T cell(s) of claim 29 or 30.

32. A method of making a modified T cell, the method comprising introducing into a T cell:
(a) a variant Cas12i polypeptide, or a nucleic acid encoding the variant Cas12i polypeptide, and a first RNA guide, or a nucleic acid encoding the first RNA guide, for inducing a deletion or an insertion in the genome of the T cell; and
(b) an exogenous nucleic acid for integration into the genome of the T cell.

33. A method of making a modified T cell, the method comprising:
(a) introducing a deletion or an insertion into the genome of a T cell using a variant Cas12i polypeptide and a first RNA guide; and
(b) introducing an exogenous nucleic acid into the genome of the T cell.

34. The method of claim 32 or 33, wherein the variant Cas12i polypeptide is a variant Cas12i2 polypeptide.

35. The method of any one of claims 32 to 34, wherein the variant Cas12i polypeptide comprises a sequence having at least 90% identity to a sequence of any one of SEQ ID NOs: 3-7.

36. The method of any one of claims 32 to 35, wherein the variant Cas12i polypeptide comprises a sequence of any one of SEQ ID NOs: 3-7.

37. The method of any one of claims 32 to 36, wherein the integration of the exogenous nucleic acid is within 100 nucleotides of a 5′-NTTN-3′ sequence.

38. The method of any one of claims 32 to 37, wherein the first RNA guide comprises a spacer sequence specific to a TRAC gene or to a B2M gene.

39. The method of claim 38, wherein the deletion or the insertion is introduced into the TRAC gene or the B2M gene.

40. The method of any claim 38 or 39, wherein the deletion or the insertion is in one or both alleles of the TRAC gene or the B2M gene.

41. The method of any one of claims 38 to 40, wherein the deletion or the insertion inhibits or decreases expression of the TRAC gene or the B2M gene.

42. The method of any one of claims 37 to 41, wherein the deletion or the insertion is within 100 nucleotides of a 5′-NTTN-3′ sequence.

43. The method of any one of claims 38 to 42, wherein the exogenous nucleic acid is integrated into the TRAC gene or the B2M gene.

44. The method of any one of claims 32 to 43, wherein the exogenous nucleic acid comprises a sequence encoding a protein.

45. The method of any one of claims 32 to 44, wherein the exogenous nucleic acid comprises a regulatory sequence operably linked to the sequence encoding the protein.

46. The method of claim 44 or 45, wherein the protein is a chimeric antigen receptor (CAR).

47. The method of claim 46, wherein the CAR comprises a single chain antibody (scFv), a transmembrane domain, an intracellular signaling domain, and, optionally, a hinge domain between the scFv and the transmembrane domain.

48. The method of claim 47, wherein the intracellular signaling domain comprises a CD3ζ signaling domain.

49. The method of claim 47 or 48, wherein the intracellular signaling domain further comprises a signaling domain selected from the group consisting of CD28, 4-1BB, ICOS, OX40, CD27, p56-lck, or a combination of two or more thereof.

50. The method of any one of claims 46 to 49, wherein the CAR comprises a scFv that is specific for CD19, CD22, BCMA, HER2, IL13Ra2, CD123, FAP, VEGFR-2, ganglioside GD2, EGFRvIII, mesothelin, or EphA.

51. The method of any one of claims 32 to 50, wherein the exogenous nucleic acid is integrated into the genome of the T cell by homology directed repair (HDR).

52. The method of any one of claims 32 to 51, wherein the exogenous nucleic acid is included in a vector.

53. The method of claim 52, wherein the vector is a viral vector.

54. The method of claim 53, wherein the viral vector is a lentiviral vector or an AAV vector.

55. The method of any one of claims 32 to 54, wherein the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the TRAC gene.

56. The method of any one of claims 32 to 54, wherein the first RNA guide comprises a spacer sequence specific to a TRAC gene and the method further comprises use of a second RNA guide or a nucleic acid encoding the second guide RNA, wherein the second guide RNA comprises a spacer sequence specific to a B2M gene, and wherein the exogenous nucleic acid is integrated into the B2M gene.

57. A modified T cell generated by a method of any one of claims 32 to 56.

58. The modified T cell of claim 57, which is a human T cell or is derived from a human cell or a human T cell.

59. A composition or formulation comprising one or more modified T cell(s) of claim 57 or 58.
